(19) United States
(12) Patent Application Publication
(10) Pub. No.: US 2026/0256938 A1
(43) Pub. Date: Sep. 3, 2026

(54) TARGETED DELIVERY TO SCHWANN CELLS AND TREATMENT METHODS IN SCHWANN CELL-RELATED DISEASES

(71) Applicant: POTENTIA LTD, Tel Aviv-Yafo (IL)

(72) Inventors: Hila EPSTEIN-BARASH, Tel Aviv-Yafo (IL); Oren BOGIN, Tel Aviv-Yafo (IL); Barak AZMON, Tel Aviv-Yafo (IL); Yair ALSTER, Tel Aviv-Yafo (IL)

(21) Appl. No.: 18/993,070

(22) PCT Filed: Jul. 11, 2023
(86) PCT No.: PCT/IB2023/000418

§ 371 Date: Jan. 10, 2025

Related U.S. Application Data
(60) Provisional application No. 63/388,543, filed on Jul. 12, 2022.

(51) Int. Cl. A61K 47/68

(52) U.S. Cl. CPC A61K 47/6849; A61K 47/6807

(57) ABSTRACT

Disclosed herein are drug conjugates comprising targeting moieties conjugated to drug molecules, wherein the targeting moieties bind to receptors expressed on Schwann cells to mediate targeted delivery of the drug conjugate to the Schwann cell, and wherein the drug molecules modify expression or activity of disease-associated molecules in the Schwann cells. Further disclosed herein are methods of targeted delivery of a drug to the Schwann cells.

### CROSS-REFERENCE

This application claims the benefit of U.S. Provisional Application No. 63/388,543 filed Jul. 12, 2022, which is incorporated herein by reference in its entirety.

### BACKGROUND

Schwann cells, also called neurilemma cells, produce the myelin sheath around neuronal axons in the peripheral nervous system. They play a key role in the pathology of various inflammatory, metabolic, or hereditary neuropathies (see, e.g., Lehmann and Höke, CNS Neurol Disord Drug Targets. 2010 Dec. 9 (6): 801-806; Kamil K, et al. Front. Neurol. 2019, 10:87 which are incorporated by reference herein in its entirety).

Some peripheral neuropathies are associated with abnormal protein or gene expression in Schwann cells, and suppression or regulation of such protein or gene expression in Schwann cells has been contemplated to treat those peripheral neuropathies. However, the drug delivery methods in the current practice, such as deliveries via nanoparticle and AAV possess disadvantages including nonspecific targeting, reduced targeting efficiency or safety concerns. As such, there exists a need to specifically target Schwann cells to manage, treat, or alleviate the large repertoire of Schwann cell-associated diseases.

### SUMMARY OF DISCLOSURE

Recognizing the need for a more effective and more targeted delivery of therapeutic compositions to Schwann cells, the present disclosure, in one aspect, provides a drug conjugate comprising a targeting moiety conjugated to a drug molecule, wherein the targeting moiety binds to a receptor expressed on a Schwann cell to mediate targeted delivery of the drug conjugate to the Schwann cell, and wherein the drug molecule modifies expression or activity of a disease-associated molecule in the Schwann cell.

In some instances, the receptor is selected from a group consisting of a leprosy receptor (e.g., laminin 2, P0 protein), gliomedin, or cell adhesion molecule (Cadm). In some instances, the receptor is gliomedin. In some instances, the receptor is Cadm. In some instances, the receptor is a leprosy receptor.

In some instances, the targeting moiety is an antibody or antigen binding fragment thereof or a ligand molecule. In some instances, the antibody or antigen binding fragment thereof comprises monovalent Fab′, divalent Fab2, single-chain variable fragment (scFv), diabody, minibody, nanobody, single-domain antibody (sdAb), or camelid antibody or binding fragment or antibody-mimetic thereof. In some instances, the targeting moiety is a ligand molecule, and wherein the ligand molecule comprises a peptide, a glycoprotein, a carbohydrate moiety, a dendrimer, or a synthetic small molecule.

In some instances, the drug molecule comprises a peptide or a small molecule. In some instances, the drug molecule comprises a double-stranded RNAi molecule or a single-stranded antisense oligonucleotide.

In some instances, the drug molecule is conjugated with the targeting moiety via a linker. In some instances, the linker is a cleavable linker or a non-cleavable linker. In some instances, the drug molecule to the targeting moiety ratio is 1:1, 2:1, or 1:2.

In some aspects, provided herein is a pharmaceutical composition comprising the drug conjugate described herein and a pharmaceutically acceptable excipient. In some instances, the pharmaceutical composition is formulated for parenteral, intravenous, subcutaneous, intrathecal, or intrasciatic injection.

In some aspects, provided herein is a method of targeted delivery of a drug to a Schwann cell, comprising contacting the drug conjugate described herein or the pharmaceutical composition described herein to the Schwann cell, wherein the drug molecule comprises the drug or a precursor of the drug. In some instances, the drug or the precursor of the drug is internalized into the Schwann cell upon binding of the targeting moiety to the receptor on the Schwann cell and exerts biological activities of the drug or the precursor of the drug.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The application contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIGS. 1A-1F illustrate the internalization of some targeting moieties, including anti-gliomedin antibody (mAb94), into Schwann cells in vitro. FIG. 1A illustrates rat Schwann cell cultures incubated with pre-labeled (in red) anti-nectin-like protein 4 (Necl4, also known as Cadm4) antibody (mAb244/5). FIG. 1B illustrates rat Schwann cell cultures incubated with pre-labeled (in red) mouse anti-gliomedin antibody (mAb94). FIG. 1C illustrates rat Schwann cell cultures incubated with pre-labeled (in red) mouse anti-myelin-associated glycoprotein (MAG) antibody (mAb513). FIG. 1D illustrates the internalization of anti-gliomedin antibody (mAb94) in rat Schwann cell cultures for different durations of incubation. FIG. 1E illustrates the co-localization of internalized anti-gliomedin antibody (mAb94) and lysosome markers in rat Schwann cell cultures. FIG. 1F illustrates the internalization of anti-gliomedin antibody (mAb94) covalently conjugated with Cyanine5 NHS-Ester in rat Schwann cell cultures.

FIGS. 2A-2C illustrate anti-gliomedin antibody (mAb94) specifically internalized into Schwann cells in mixed cultures and recognized both rat and human gliomedin. FIG. 2A illustrates anti-gliomedin antibody (mAb94) specifically internalized into wild type Schwann cells in mixed cultures with neurons and Schwann cells, but not the mutant Schwann cells without gliomedin expression. FIG. 2B illustrates anti-gliomedin antibody (mAb94) specifically internalized into wild type Schwann cells in mixed cultures with neurons and Schwann cells, but not the mutant Schwann cells without gliomedin expression. FIG. 2C illustrates anti-gliomedin antibody (mAb94) specifically bound to wild type Schwann cells in mixed cultures with neurons, Schwann cells, and fibroblasts, but not the mutant Schwann cells without gliomedin expression.

FIG. 3 illustrates anti-gliomedin antibody (mAb94) internalized into COS7 cells transfected with either rat or human gliomedin.

FIGS. 4A-4E illustrate the internalization of anti-gliomedin antibody (mAb94) into Schwann cells in vivo and its distribution among different organs. FIG. 4A illustrates sciatic nerves collected from mice #1-3 injected with 650-mAb94 antibody and mice #7-9 injected with control 650-IgG. Both groups were sacrificed 24 hours after injections. FIG. 4B illustrates sciatic nerves collected from mice #4-6 injected with 650-mAb94 antibody and mice #10-12 injected with control 650-IgG. Both groups were sacrificed 48 hours after injections. FIG. 4C illustrates a comparison between the sciatic nerve from a mouse injected with control 650-IgG (#12) and an un-injected mouse (#13). FIG. 4D illustrates fluorescent images for Cy5 labeled anti-gliomedin antibody (mAb94) (far red) in different organs 24 hours after intravenous injection. FIG. 4E illustrates fluorescent images for Cy5 labeled anti-gliomedin antibody (mAb94) (far red) in different organs 48 hours after intravenous injection.

### DETAILED DESCRIPTION

Provided herein are drug conjugates comprising targeting moieties conjugated to drug molecules, wherein the targeting moieties bind to receptors expressed on Schwann cells to mediate targeted delivery of the drug conjugate to the Schwann cell, and wherein the drug molecules modify expression or activity of disease-associated molecules in the Schwann cells. Further disclosed herein are methods of targeted delivery of a drug to the Schwann cells. Also disclosed herein are methods of delivering of a drug to Schwann cell by conjugating the drug to a targeting moiety that specifically or preferentially target the Schwann cell. Also disclosed herein are methods of treating or preventing a peripheral demyelinating disease or a peripheral neuropathy, or alleviating or reducing symptoms of the peripheral demyelinating disease or the peripheral neuropathy in a subject in need thereof.

### Conjugates

### Targeting Moieties

Historically, the treatment of peripheral nervous system (PNS) disorders associated with, e.g., axonal loss, dysfunction of the neuronal axon, or abnormal axon-Schwann cell interaction, has focused on targeting axons, but the treatment has been shown with poor clinical outcomes. For certain PNS disorders where the axonal dysfunction is associated with the loss of Schwann cells, or the PNS disorders that are directly or indirectly related to the dysfunction of the Schwann cells, specific or preferred targeting of drugs to the Schwann cell can increase the efficacy and outcome of the treatment.

However, the specificity of the manipulation and/or modification of Schwann cells is a main roadblock to advance Schwann cell therapies. To meet such a need, provided herein in some instances is the target moiety that specifically directs cargoes to Schwann cells.

In some aspects, the targeting moiety disclosed herein binds to a receptor expressed on a Schwann cell. In some aspects, the targeting moiety disclosed herein binds to a portion of receptor complex expressed on a Schwann cell. In some instances, the receptor is a transmembrane molecule comprising an extracellular domain, and the targeting moiety binds to the extracellular domain of the receptor. In some instances, the receptor complex comprises an extracellular molecule that forms a complex with the receptor molecule (e.g., membrane bound receptor via transmembrane domain) directly or indirectly. In some instances, the targeting moiety binds to the extracellular molecule of the complex. In some instances, the targeting moiety binds to a cell adhesion molecule that mediates the interaction between myelinating Schwann cells and the axons they ensheath. In some instances, the receptor is expressed in the myelinated Schwann cells. In some instances, the receptor is highly expressed at the edge of myelin cells. In some instances, the receptor mediates the interaction of axon and glial cells, or mediates axon-glial contact. In some instances, the receptor mediates the pathogen (or a portion thereof) uptake to the Schwan cells in certain disease conditions. As such, as used herein, the targeting moiety binding to a receptor also includes a targeting moiety that binds to a portion of a receptor complex that are naturally present in healthy or pathological conditions of Schwann cells.

In some instances, the targeting moiety disclosed herein binds to a leprosy receptor. In specific instances, the targeting moiety disclosed herein binds to laminin 2. In specific instances, the targeting moiety disclosed herein binds to the C-terminal region of laminin 2. In some instances, the targeting moiety disclosed herein binds to the α2-subunit of laminin 2. In some instances, the targeting moiety disclosed herein binds to G domain of the α2-subunit of laminin 2. In some instances, the targeting moiety disclosed herein binds to G1 domain of the α2-subunit of laminin 2. In some instances, the targeting moiety disclosed herein binds to G2 domain of the α2-subunit of laminin 2. In some instances, the targeting moiety disclosed herein binds to G3 domain of the α2-subunit of laminin 2. In some instances, the targeting moiety disclosed herein binds to G4 domain of the α2-subunit of laminin 2. In some instances, the targeting moiety disclosed herein binds to G5 domain of the α2-subunit of laminin 2. In some instances, the targeting moiety disclosed herein binds to the β1-subunit of laminin 2. In some instances, the targeting moiety disclosed herein binds to the γ1-subunit of laminin 2. In some instances, the targeting moiety disclosed herein binds amino acids 2901-3106 of lamin-2. In other instances, the targeting moiety disclosed herein binds to P0 protein (myelin protein zero). In some instances, the targeting moiety disclosed herein binds to an N-terminal extracellular immunoglobulin (Ig)-like domain of P0 protein. In some instances, the targeting moiety disclosed herein binds to the N-linked oligosaccharide in the extracellular domain of the leprosy receptor or any subunits thereof, and/or the addition of sulfate, acyl, and phosphate groups.

In some instances, the targeting moiety disclosed herein is an antibody or antigen binding fragment thereof which binds to a leprosy receptor. In specific instances, the targeting moiety disclosed herein is an antibody or antigen binding fragment thereof which binds to laminin 2. In specific instances, the targeting moiety disclosed herein is an antibody or antigen binding fragment thereof which binds to the C-terminal region of laminin 2. In some instances, the targeting moiety disclosed herein is an antibody or antigen binding fragment thereof which binds to the α2-subunit of laminin 2. In some instances, the targeting moiety disclosed herein is an antibody or antigen binding fragment thereof which binds to G domain of the α2-subunit of laminin 2. In some instances, the targeting moiety disclosed herein is an antibody or antigen binding fragment thereof which binds to G1 domain of the α2-subunit of laminin 2. In some instances, the targeting moiety disclosed herein is an antibody or antigen binding fragment thereof which binds to G2 domain of the α2-subunit of laminin 2. In some instances, the targeting moiety disclosed herein is an antibody or antigen binding fragment thereof which binds to G3 domain of the α2-subunit of laminin 2. In some instances, the targeting moiety disclosed herein is an antibody or antigen binding fragment thereof which binds to G4 domain of the α2-subunit of laminin 2. In some instances, the targeting moiety disclosed herein is an antibody or antigen binding fragment thereof which binds to G5 domain of the α2-subunit of laminin 2. In some instances, the targeting moiety disclosed herein is an antibody or antigen binding fragment thereof which binds to the β1-subunit of laminin 2. In some instances, the targeting moiety disclosed herein is an antibody or antigen binding fragment thereof which binds to the γ1-subunit of laminin 2. In some instances, the targeting moiety disclosed herein is an antibody or antigen binding fragment thereof which binds amino acids 2901-3106 of lamin-2. In other instances, the targeting moiety disclosed herein is an antibody or antigen binding fragment thereof which binds to P0 protein (myelin protein zero). In some instances, the targeting moiety disclosed herein is an antibody or antigen binding fragment thereof which binds to an N-terminal extracellular immunoglobulin (Ig)-like domain of P0 protein. In some instances, the targeting moiety disclosed herein is an antibody or antigen binding fragment thereof which binds to the N-linked oligosaccharide in the extracellular domain of the leprosy receptor or any subunits thereof, and/or the addition of sulfate, acyl, and phosphate groups.

In some instances, the targeting moiety disclosed herein is a commercially available antibody that binds to laminin-2. In some instances, the targeting moiety disclosed herein is anti-laminin antibody ABIN7439129. In some instances, the targeting moiety disclosed herein is anti-laminin alpha-2 monoclonal antibody (Thermo Fisher, #CL3450).

In some instances, the targeting moiety disclosed herein binds to a gliomedin. In some instances, the targeting moiety disclosed herein binds to the extracellular region of a gliomedin. In some instances, the targeting moiety disclosed herein binds to the C-terminal region of a gliomedin. In some instances, the targeting moiety disclosed herein binds to a portion of the C-terminal region of a gliomedin. In some instances, the targeting moiety disclosed herein binds to the coiled-coil domain of a gliomedin. In some instances, the targeting moiety disclosed herein binds to the interrupted collagen repeats domain of a gliomedin. In some instances, the targeting moiety disclosed herein binds to the olfactomedin domain of a gliomedin. In some instances, the targeting moiety disclosed herein binds to amino acid residues 273-287 of rat gliomedin NP_852047.2 (CVIPNDDTLVGRA). In some instances, the targeting moiety disclosed herein binds to amino acid residues 365-460 of a gliomedin.

In some instances, the targeting moiety disclosed herein is an antibody or antigen binding fragment thereof which binds to a gliomedin. In some instances, the targeting moiety disclosed herein is an antibody or antigen binding fragment thereof which binds to the extracellular region of a gliomedin. In some instances, the targeting moiety disclosed herein is an antibody or antigen binding fragment thereof which binds to the C-terminal region of a gliomedin. In some instances, the targeting moiety disclosed herein is an antibody or antigen binding fragment thereof which binds to a portion of the C-terminal region of a gliomedin. In some instances, the targeting moiety disclosed herein is an antibody or antigen binding fragment thereof which binds to the coiled-coil domain of a gliomedin. In some instances, the targeting moiety disclosed herein is an antibody or antigen binding fragment thereof which binds to the interrupted collagen repeats domain of a gliomedin. In some instances, the targeting moiety disclosed herein is an antibody or antigen binding fragment thereof which binds to the olfactomedin domain of a gliomedin. In some instances, the targeting moiety disclosed herein is an antibody or antigen binding fragment thereof which binds to amino acid residues 273-287 of rat gliomedin NP_852047.2 (CVIPNDDTLVGRA). In some instances, the targeting moiety disclosed herein is an antibody or antigen binding fragment thereof which binds to amino acid residues 365-460 of a gliomedin.

In some instances, the targeting moiety disclosed herein is a commercially available antibody that binds to gliomedin. In some instances, the targeting moiety disclosed herein is anti-gliomedin antibody (mAb94) disclosed in Eshed et al., Neuron, volume 47, issue 2, p215-229, which is incorporated by reference in its entirety. In some instances, the targeting moiety disclosed herein is anti-GLDN antibody NovoPro #: 175453. In some instances, the targeting moiety disclosed herein is anti-GLDN antibody Thermo Fisher #: BS-11032R.

In some instances, the targeting moiety disclosed herein binds to a cell adhesion molecule (Cadm). In some instances, the targeting moiety disclosed herein binds to Cadm1. In some instances, the targeting moiety disclosed herein binds to IgV-set domain of Cadm1 or a portion thereof. In some instances, the targeting moiety disclosed herein binds to IgC1-set domain of Cadm1 or a portion thereof. In some instances, the targeting moiety disclosed herein binds to IgI-set domain of Cadm1 or a portion thereof. In specific instances, the targeting moiety disclosed herein binds to Cadm2. In some instances, the targeting moiety disclosed herein binds to IgV-set domain of Cadm2 or a portion thereof. In some instances, the targeting moiety disclosed herein binds to IgC1-set domain of Cadm2 or a portion thereof. In some instances, the targeting moiety disclosed herein binds to IgI-set domain of Cadm2 or a portion thereof. In some instances, the targeting moiety disclosed herein binds to Cadm3. In some instances, the targeting moiety disclosed herein binds to IgV-set domain of Cadm3 or a portion thereof. In some instances, the targeting moiety disclosed herein binds to IgC1-set domain of Cadm3 or a portion thereof. In some instances, the targeting moiety disclosed herein binds to IgI-set domain of Cadm3 or a portion thereof. In some instances, the targeting moiety disclosed herein binds to Cadm4. In some instances, the targeting moiety disclosed herein binds to IgV-set domain of Cadm4 or a portion thereof. In some instances, the targeting moiety disclosed herein binds to IgC1-set domain of Cadm4 or a portion thereof. In some instances, the targeting moiety disclosed herein binds to IgI-set domain of Cadm4 or a portion thereof.

In some instances, the targeting moiety disclosed herein an antibody or antigen binding fragment thereof which binds to a cell adhesion molecule (Cadm). In some instances, the targeting moiety disclosed herein an antibody or antigen binding fragment thereof which binds to Cadm1. In some instances, the targeting moiety disclosed herein an antibody or antigen binding fragment thereof which binds to IgV-set domain of Cadm1 or a portion thereof. In some instances, the targeting moiety disclosed herein an antibody or antigen binding fragment thereof which binds to IgC1-set domain of Cadm1 or a portion thereof. In some instances, the targeting moiety disclosed herein an antibody or antigen binding fragment thereof which binds to IgI-set domain of Cadm1 or a portion thereof. In specific instances, the targeting moiety disclosed herein an antibody or antigen binding fragment thereof which binds to Cadm2. In some instances, the targeting moiety disclosed herein an antibody or antigen binding fragment thereof which binds to IgV-set domain of Cadm2 or a portion thereof. In some instances, the targeting moiety disclosed herein an antibody or antigen binding fragment thereof which binds to IgC1-set domain of Cadm2 or a portion thereof. In some instances, the targeting moiety disclosed herein an antibody or antigen binding fragment thereof which binds to IgI-set domain of Cadm2 or a portion thereof. In some instances, the targeting moiety disclosed herein an antibody or antigen binding fragment thereof which binds to Cadm3. In some instances, the targeting moiety disclosed herein an antibody or antigen binding fragment thereof which binds to IgV-set domain of Cadm3 or a portion thereof. In some instances, the targeting moiety disclosed herein an antibody or antigen binding fragment thereof which binds to IgC1-set domain of Cadm3 or a portion thereof. In some instances, the targeting moiety disclosed herein an antibody or antigen binding fragment thereof which binds to IgI-set domain of Cadm3 or a portion thereof. In some instances, the targeting moiety disclosed herein an antibody or antigen binding fragment thereof which an antibody or antigen binding fragment thereof which binds to Cadm4. In some instances, the targeting moiety disclosed herein an antibody or antigen binding fragment thereof which binds to IgV-set domain of Cadm4 or a portion thereof. In some instances, the targeting moiety disclosed herein an antibody or antigen binding fragment thereof which binds to IgC1-set domain of Cadm4 or a portion thereof. In some instances, the targeting moiety disclosed herein an antibody or antigen binding fragment thereof which binds to IgI-set domain of Cadm4 or a portion thereof.

In some instances, the targeting moiety disclosed herein is a commercially available antibody that binds to Cadm1. In some instances, the targeting moiety disclosed herein is a commercially available antibody that binds to Cadm2. In some instances, the targeting moiety disclosed herein is a commercially available antibody that binds to Cadm3. In some instances, the targeting moiety disclosed herein is a commercially available antibody that binds to Cadm4. In some instances, the targeting moiety disclosed herein is anti-Cadm4 mAb244/5 (NeuroMAB). In some instances, the targeting moiety disclosed herein is Necl4-Fc antibody from Eshed et al., Neuron, volume 47, issue 2, p 215-229. In some instances, the targeting moiety disclosed herein is anti-SynCAM4 Antibody (Biolegand, #833302). In some instances, the targeting moiety disclosed herein is IGSF4C/SynCAM4 Antibody (rndsystems, #MAB41642).

In some instances, the targeting moiety directs the conjugate to Schwann cells, and the conjugates are internalized upon the binding of the targeting moiety to the receptor, which is about 24 hours, about 36 hours, about 48 hours, about 60 hours, or about 72 hours after contacting the Schwann cell. In some instances, the targeting moiety directs the conjugate to Schwann cells, and the conjugates are internalized upon the binding of the targeting moiety to the receptor, which is within about 24 hours, about 36 hours, about 48 hours, about 60 hours, or about 72 hours after contacting the Schwann cell. In some instances, the targeting moiety disclosed herein directs and internalizes the conjugate disclosed herein more than 1, 2, 3, 4, 5, 6, 7, 8, 9, 10, 11, 12, 13, 14, 15, 16, 17, 18, 19, 20, 21, 22, 23, or 24 hours after contacting the Schwann cell. In some instances, the targeting moiety disclosed herein directs and internalizes the conjugate disclosed herein less than 72, 73, 74, 75, 76, 77, 78, 79, 80, 81, 82, 83, 84, 85, 86, 87, 88, 89, 90, 91, 92, 93, 94, 95, or 96 hours after contacting the Schwann cell.

In some instances, the drug molecules disclosed herein are released after being internalized into the Schwann cells. In some instances, the drug molecules disclosed herein are released after the conjugates disclosed herein enter a lysosome of the Schwann cells. In some instances, the drug molecules disclosed herein are released to the cytoplasm of the Schwann cells. In some instances, the drug molecules disclosed herein are released to the nucleus of the Schwann cells.

In some aspects, the targeting moiety disclosed herein is an antibody or antigen binding fragment thereof. In some instances, the antibody or antigen binding fragment thereof comprises monovalent Fab′, divalent Fab2, single-chain variable fragment (scFv), diabody, minibody, nanobody, single-domain antibody (sdAb), or camelid antibody or binding fragment thereof. In other instances, the targeting moiety disclosed herein is (scFv) 2, Fab, Fab′, F(ab′) 2, Fv, dAb, Fd fragments, diabodies, F(ab′) 3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin and mini-proteins. In other instances, the targeting moiety disclosed herein is variants and derivatives of antibodies include antibody functional fragments that retain the ability to bind to the receptor expressed on a Schwann cell. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab′ and an additional portion of the heavy chain through the hinge region); F(ab′)2 (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen binding domains may be directed towards the same or different epitopes); and a tetrabody (e.g., a tetramerized scFv, formed in a manner similar to a diabody, but in which four antigen-binding domains are created in a single complex; the four antigen binding domains may be directed towards the same or different epitopes). In some aspects, the targeting moiety disclosed herein is an antibody or antigen binding fragment thereof with mono-specificity, bi-specificity, tri-specificity, or multi-specificity. In other aspects, the targeting moiety disclosed herein is a combination of one or more of the above-mentioned antibodies or antigen binding fragments thereof.

In some aspects, the targeting moiety disclosed herein is a ligand molecule. In some instances, the ligand molecule is recognized by Schwann cells and the conjugates disclosed herein are internalized into the Schwann cells. In some instances, the ligand molecule comprises a peptide, a glycoprotein, a carbohydrate moiety, a dendrimer, or a synthetic small molecule. In some instances, the ligand molecule described herein binds to laminin 2. In some instances, the ligand molecule described herein binds to P0 protein. In some instances, the ligand molecule described herein binds to gliomedin. In some instances, the ligand molecule described herein binds to Cadm1. In some instances, the ligand molecule described herein binds to Cadm2. In some instances, the ligand molecule described herein binds to Cadm3. In some instances, the ligand molecule described herein binds to Cadm4.

In other aspects, the targeting moiety disclosed herein is a combination of one or more of the above-mentioned antibodies, antigen binding fragments thereof, or ligand molecules.

In some instances, the targeting moiety disclosed herein comprises at least a portion of neurofascin-186 (NF186) or a derivative thereof. In some instances, the targeting moiety disclosed herein comprises one or more gliomedin-binding domain of NF186.

In some instances, the targeting moiety disclosed herein comprises at least 1%, 5%, 10%, 20%, 30%, 40%, 50%, or 60% of full length NF186 without significantly affecting the binding affinity to gliomedin. In some instances, the targeting moiety disclosed herein comprises no greater than 10%, 20%, 30%, 40%, 50%, 60%, 70%, 80%, 90%, 95%, or 99% of full length NF186 without significantly affecting the binding affinity to gliomedin. In some instances, the targeting moiety disclosed herein comprises a portion of NF186 with one or more conservative amino acid substitutions without significantly affecting the binding affinity to gliomedin. In some instances, the targeting moiety disclosed herein comprises a portion of NF186 with one or more non-conservative amino acid substitutions without significantly affecting the binding affinity to gliomedin. In some instances, the targeting moiety disclosed herein comprises a portion of NF186 with one or more substitution with a basic, an acidic, an aromatic, an aliphatic, an acid amide, a cyclic, or a sulfur-containing amino acid without significantly affecting the binding affinity to gliomedin. In some instances, the targeting moiety disclosed herein comprises a portion of NF186 with one or more substitution with a standard amino acid or a non-standard amino acid without significantly affecting the binding affinity to gliomedin. In some instances, the targeting moiety disclosed herein comprises a portion of NF186 with one or more substitution with an alpha, a beta, a gamma, or a delta amino acid without significantly affecting the binding affinity to gliomedin. In some instances, the targeting moiety disclosed herein comprises a portion of NF186 with one or more substitution with a positively-charged, negatively-charged, or neutral amino acid without significantly affecting the binding affinity to gliomedin. In some instances, the targeting moiety disclosed herein comprises a portion of NF186 with one or more substitution with a polar or non-polar amino acid without significantly affecting the binding affinity to gliomedin. In some instances, the targeting moiety disclosed herein comprises a portion of NF186 with one or more amino acid deletions without significantly affecting the binding affinity to gliomedin. In some instances, the targeting moiety disclosed herein comprises a portion of NF186 with one or more amino acid insertions without significantly affecting the binding affinity to gliomedin. In some instances, the targeting moiety disclosed herein comprises a portion of NF186 and an amino acid sequence which is at least partly the reverse of the amino acid sequence of the corresponding portion of NF186, without significantly affecting the binding affinity to gliomedin. In some instances, the targeting moiety disclosed herein comprises a portion of NF186, and wherein at least one of the amino acids from the original portion of NF186 is replaced by a stereoisomer (e.g., D-stereoisomer) of that amino acid, without significantly affecting the binding affinity to gliomedin. In some instances, the targeting moiety disclosed herein comprises a portion of NF186, and is extended at one or both ends thereof with one or more groups, such as D-amino acids, without significantly affecting the binding affinity to gliomedin. In some instances, the targeting moiety disclosed herein comprises NF186 with a combination of one or more of the above-mentioned modifications without significantly affecting the binding affinity to gliomedin.

In some instances, the targeting moiety disclosed herein comprises a recombinant NF186 peptide or a derivative thereof. In some instances, the targeting moiety disclosed herein comprises a gliomedin-binding domain of recombinant NF186. In some instances, the targeting moiety disclosed herein comprises at least 1%, 5%, 10%, 20%, 30%, 40%, 50%, or 60% of full length recombinant NF186 without significantly affecting the binding affinity to gliomedin. In some instances, the targeting moiety disclosed herein comprises no greater than 10%, 20%, 30%, 40%, 50%, 60%, 70%, 80%, 90%, 95%, or 99% of full length recombinant NF186 without significantly affecting the binding affinity to gliomedin. In some instances, the targeting moiety disclosed herein comprises a portion of recombinant NF186 with one or more conservative amino acid substitutions without significantly affecting the binding affinity to gliomedin. In some instances, the targeting moiety disclosed herein comprises a portion of recombinant NF186 with one or more non-conservative amino acid substitutions without significantly affecting the binding affinity to gliomedin. In some instances, the targeting moiety disclosed herein comprises a portion of recombinant NF186 with one or more substitution with a basic, an acidic, an aromatic, an aliphatic, an acid amide, a cyclic, or a sulfur-containing amino acid without significantly affecting the binding affinity to gliomedin. In some instances, the targeting moiety disclosed herein comprises a portion of recombinant NF186 with one or more substitution with a standard amino acid or a non-standard amino acid without significantly affecting the binding affinity to gliomedin. In some instances, the targeting moiety disclosed herein comprises a portion of recombinant NF186 with one or more substitution with an alpha, a beta, a gamma, or a delta amino acid without significantly affecting the binding affinity to gliomedin. In some instances, the targeting moiety disclosed herein comprises a portion of recombinant NF186 with one or more substitution with a positively-charged, negatively-charged, or neutral amino acid without significantly affecting the binding affinity to gliomedin. In some instances, the targeting moiety disclosed herein comprises a portion of recombinant NF186 with one or more substitution with a polar or non-polar amino acid without significantly affecting the binding affinity to gliomedin. In some instances, the targeting moiety disclosed herein comprises a portion of recombinant NF186 with one or more amino acid deletions without significantly affecting the binding affinity to gliomedin. In some instances, the targeting moiety disclosed herein comprises a portion of recombinant NF186 with one or more amino acid insertions without significantly affecting the binding affinity to gliomedin. In some instances, the targeting moiety disclosed herein comprises a portion of recombinant NF186 and an amino acid sequence which is at least partly the reverse of the amino acid sequence of the corresponding portion of recombinant NF186 without significantly affecting the binding affinity to gliomedin. In some instances, the targeting moiety disclosed herein comprises a portion of recombinant NF186, and wherein at least one of the amino acids from the original portion of recombinant NF186 is replaced by a stereoisomer (e.g., D-stereoisomer) of that amino acid without significantly affecting the binding affinity to gliomedin. In some instances, the targeting moiety disclosed herein comprises a portion of recombinant NF186, and is extended at one or both ends thereof with one or more groups, such as D-amino acids, without significantly affecting the binding affinity to gliomedin. In some instances, the targeting moiety disclosed herein comprises recombinant NF186 with a combination of one or more of the above-mentioned modifications without significantly affecting the binding affinity to gliomedin.

In some instances, the targeting moiety disclosed herein binds to a neural-specific extracellular matrix protein that binds to a receptor expressed on Schwann cells. In some instances, the targeting moiety disclosed herein binds to laminin 2. In some instances, the targeting moiety disclosed herein binds to the α2-subunit of laminin 2. In some instances, the targeting moiety disclosed herein binds to G domain of the α2-subunit of laminin 2. In some instances, the targeting moiety disclosed herein binds to G1 domain of the α2-subunit of laminin 2. In some instances, the targeting moiety disclosed herein binds to G2 domain of the α2-subunit of laminin 2. In some instances, the targeting moiety disclosed herein binds to G3 domain of the α2-subunit of laminin 2. In some instances, the targeting moiety disclosed herein binds to G4 domain of the α2-subunit of laminin 2. In some instances, the targeting moiety disclosed herein binds to G5 domain of the α2-subunit of laminin 2. In some instances, the targeting moiety disclosed herein binds to the β1-subunit of laminin 2. In some instances, the targeting moiety disclosed herein binds to the γ1-subunit of laminin 2.

In some instances, the targeting moiety disclosed herein comprises at least a portion of glycolipid PGL-1 or a derivative thereof. In some instances, the targeting moiety disclosed herein comprises the unique trisaccharide of PGL-1 (3,6-di-O-methylglucose linked α-1→4 to 2,3-di-O-methylrhamnose linked β-1→2 to 3-O-methylrhamnose).

In some instances, the targeting moiety disclosed herein comprises at least a portion of ML-LBP21 (also known histone-like protein/Hlp) or a derivative thereof. In some instances, the targeting moiety disclosed herein comprises a laminin-2-binding domain of ML-LBP21. In some instances, the targeting moiety disclosed herein comprises at least 1%, 5%, 10%, 20%, 30%, 40%, 50%, or 60% of full length ML-LBP21 without significantly affecting the binding affinity to laminin-2. In some instances, the targeting moiety disclosed herein comprises no greater than 10%, 20%, 30%, 40%, 50%, 60%, 70%, 80%, 90%, 95%, or 99% of full length ML-LBP21 without significantly affecting the binding affinity to laminin-2. In some instances, the targeting moiety disclosed herein comprises a portion of ML-LBP21 with one or more conservative amino acid substitutions without significantly affecting the binding affinity to laminin-2. In some instances, the targeting moiety disclosed herein comprises a portion of ML-LBP21 with one or more non-conservative amino acid substitutions without significantly affecting the binding affinity to laminin-2. In some instances, the targeting moiety disclosed herein comprises a portion of ML-LBP21 with one or more substitution with a basic, an acidic, an aromatic, an aliphatic, an acid amide, a cyclic, or a sulfur-containing amino acid without significantly affecting the binding affinity to laminin-2. In some instances, the targeting moiety disclosed herein comprises a portion of ML-LBP21 with one or more substitution with a standard amino acid or a non-standard amino acid without significantly affecting the binding affinity to laminin-2. In some instances, the targeting moiety disclosed herein comprises a portion of ML-LBP21 with one or more substitution with an alpha, a beta, a gamma, or a delta amino acid without significantly affecting the binding affinity to laminin-2. In some instances, the targeting moiety disclosed herein comprises a portion of ML-LBP21 with one or more substitution with a positively-charged, negatively-charged, or neutral amino acid without significantly affecting the binding affinity to laminin-2. In some instances, the targeting moiety disclosed herein comprises a portion of ML-LBP21 with one or more substitution with a polar or non-polar amino acid without significantly affecting the binding affinity to laminin-2. In some instances, the targeting moiety disclosed herein comprises a portion of ML-LBP21 with one or more amino acid deletions without significantly affecting the binding affinity to laminin-2. In some instances, the targeting moiety disclosed herein comprises a portion of ML-LBP21 with one or more amino acid insertions without significantly affecting the binding affinity to laminin-2. In some instances, the targeting moiety disclosed herein comprises a portion of ML-LBP21 and an amino acid sequence which is at least partly the reverse of the amino acid sequence of the corresponding portion of ML-LBP21, without significantly affecting the binding affinity to laminin-2. In some instances, the targeting moiety disclosed herein comprises a portion of ML-LBP21, and wherein at least one of the amino acids from the original portion of ML-LBP21 is replaced by a stereoisomer (e.g., D-stereoisomer) of that amino acid, without significantly affecting the binding affinity to laminin-2. In some instances, the targeting moiety disclosed herein comprises a portion of ML-LBP21, and is extended at one or both ends thereof with one or more groups, such as D-amino acids, without significantly affecting the binding affinity to laminin-2. In some instances, the targeting moiety disclosed herein comprises ML-LBP21 with a combination of one or more of the above-mentioned modifications without significantly affecting the binding affinity to laminin-2.

In some instances, the targeting moiety disclosed herein comprises a recombinant ML-LBP21 peptide or a derivative thereof. In some instances, the targeting moiety disclosed herein comprises a laminin-2-binding domain of recombinant ML-LBP21. In some instances, the targeting moiety disclosed herein comprises at least 1%, 5%, 10%, 20%, 30%, 40%, 50%, or 60% of full length recombinant ML-LBP21 without significantly affecting the binding affinity to laminin-2. In some instances, the targeting moiety disclosed herein comprises no greater than 10%, 20%, 30%, 40%, 50%, 60%, 70%, 80%, 90%, 95%, or 99% of full length recombinant ML-LBP21 without significantly affecting the binding affinity to laminin-2. In some instances, the targeting moiety disclosed herein comprises a portion of recombinant ML-LBP21 with one or more conservative amino acid substitutions without significantly affecting the binding affinity to laminin-2. In some instances, the targeting moiety disclosed herein comprises a portion of recombinant ML-LBP21 with one or more non-conservative amino acid substitutions without significantly affecting the binding affinity to laminin-2. In some instances, the targeting moiety disclosed herein comprises a portion of recombinant ML-LBP21 with one or more substitution with a basic, an acidic, an aromatic, an aliphatic, an acid amide, a cyclic, or a sulfur-containing amino acid without significantly affecting the binding affinity to laminin-2. In some instances, the targeting moiety disclosed herein comprises a portion of recombinant ML-LBP21 with one or more substitution with a standard amino acid or a non-standard amino acid without significantly affecting the binding affinity to laminin-2. In some instances, the targeting moiety disclosed herein comprises a portion of recombinant ML-LBP21 with one or more substitution with an alpha, a beta, a gamma, or a delta amino acid without significantly affecting the binding affinity to laminin-2. In some instances, the targeting moiety disclosed herein comprises a portion of recombinant ML-LBP21 with one or more substitution with a positively-charged, negatively-charged, or neutral amino acid without significantly affecting the binding affinity to laminin-2. In some instances, the targeting moiety disclosed herein comprises a portion of recombinant ML-LBP21 with one or more substitution with a polar or non-polar amino acid without significantly affecting the binding affinity to laminin-2. In some instances, the targeting moiety disclosed herein comprises a portion of recombinant ML-LBP21 with one or more amino acid deletions without significantly affecting the binding affinity to laminin-2. In some instances, the targeting moiety disclosed herein comprises a portion of recombinant ML-LBP21 with one or more amino acid insertions without significantly affecting the binding affinity to laminin-2. In some instances, the targeting moiety disclosed herein comprises a portion of recombinant ML-LBP21 and an amino acid sequence which is at least partly the reverse of the amino acid sequence of the corresponding portion of recombinant ML-LBP21 without significantly affecting the binding affinity to laminin-2. In some instances, the targeting moiety disclosed herein comprises a portion of recombinant ML-LBP21, and wherein at least one of the amino acids from the original portion of recombinant ML-LBP21 is replaced by a stereoisomer (e.g., D-stereoisomer) of that amino acid without significantly affecting the binding affinity to laminin-2. In some instances, the targeting moiety disclosed herein comprises a portion of recombinant ML-LBP21, and is extended at one or both ends thereof with one or more groups, such as D-amino acids, without significantly affecting the binding affinity to laminin-2. In some instances, the targeting moiety disclosed herein comprises recombinant ML-LBP21 with a combination of one or more of the above-mentioned modifications without significantly affecting the binding affinity to laminin-2.

In some aspects, the targeting moiety disclosed herein comprises a combination of one or more of the above-mentioned molecules.

### Drug Molecules

In some aspects, the drug molecules disclosed herein modify the expression of a disease-associated molecule. In specific aspects, the overexpression of the disease-associated molecule leads to the disease. Accordingly, in some cases, the drug molecules disclosed herein reduce the transcription/RNA level of the disease-associated molecule by at least 1%, 5%, 10%, 15%, 20%, 25%, 30%, 35%, 40%, 45%, 50%, 55%, 60%, 65%, 70%, 75%, 80%, 85%, 90%, or 95%. Accordingly, in some cases, the drug molecules disclosed herein reduce the transcription/RNA level of the disease-associated molecule to a level of those of healthy individuals. Accordingly, in other cases, the drug molecules disclosed herein reduce the translation/protein level of the disease-associated molecule by at least 1%, 5%, 10%, 15%, 20%, 25%, 30%, 35%, 40%, 45%, 50%, 55%, 60%, 65%, 70%, 75%, 80%, 85%, 90%, or 95%. Accordingly, in some cases, the drug molecules disclosed herein reduce the translation/protein level of the disease-associated molecule to a level of those of healthy individuals. In specific aspects, the lack of the disease-associated molecule leads to the disease. Accordingly, in some cases, the drug molecules disclosed herein increase the transcription/RNA level of the disease-associated molecule by at least 1%, 5%, 10%, 15%, 20%, 25%, 30%, 35%, 40%, 45%, 50%, 55%, 60%, 65%, 70%, 75%, 80%, 85%, 90%, or 95%. Accordingly, in some cases, the drug molecules disclosed herein increase the transcription/RNA level of the disease-associated molecule to a level of those of healthy individuals. Accordingly, in other cases, the drug molecules disclosed herein increase the translation/protein level of the disease-associated molecule by at least 1%, 5%, 10%, 15%, 20%, 25%, 30%, 35%, 40%, 45%, 50%, 55%, 60%, 65%, 70%, 75%, 80%, 85%, 90%, or 95%. Accordingly, in some cases, the drug molecules disclosed herein increase the translation/protein level of the disease-associated molecule to a level of those of healthy individuals.

In other aspects, the drug molecules disclosed herein modify the activity of a disease-associated molecule. In specific aspects, the hyperactivity of the disease-associated molecule leads to the disease. Accordingly, in some cases, the drug molecules disclosed herein reduce the activity of the disease-associated molecule by at least 1%, 5%, 10%, 15%, 20%, 25%, 30%, 35%, 40%, 45%, 50%, 55%, 60%, 65%, 70%, 75%, 80%, 85%, 90%, or 95%. Accordingly, in some cases, the drug molecules disclosed herein reduce the activity of the disease-associated molecule to a level of those of healthy individuals. In specific aspects, the hypoactivity of the disease-associated molecule leads to the disease. Accordingly, in some cases, the drug molecules disclosed herein increase the activity of the disease-associated molecule by at least 1%, 5%, 10%, 15%, 20%, 25%, 30%, 35%, 40%, 45%, 50%, 55%, 60%, 65%, 70%, 75%, 80%, 85%, 90%, or 95%. Accordingly, in some cases, the drug molecules disclosed herein increase the activity of the disease-associated molecule to a level of those of healthy individuals.

In some instances, the drug molecule comprises a drug or a precursor form of the drug (a drug precursor or a prodrug). In some instances, the drug molecule comprises a peptide. In some instances, the drug molecule comprises a protein. Any suitable peptide or protein may be used as a drug molecule to modify the expression or activity of the disease-associated molecule, as described herein. In some cases, the drug molecule comprises an enzyme. In some cases, peptides or proteins may be produced, synthesized, and/or derivatized using several methodologies, e.g. phage displayed peptide libraries, one-bead one-compound peptide libraries, directed evolution, or positional scanning synthetic peptide combinatorial libraries. In some instances, the drug molecule comprises peptide or protein of about 2-25 amino acids, about 2-20 amino acids, about 2-15 amino acids, about 2-10 amino acids, or about 2-5 amino acids. The peptide or protein may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some cases, the peptide may be linear; in other instances, the peptide may be cyclic, e.g. bicyclic.

In some instances, the drug molecule comprises a small molecule. In some instances, the small molecule is a modulator of the disease-associated molecule. In some instances, the small molecule is an agonist of the disease-associated molecule. In some instances, the small molecule is an antagonist of the disease-associated molecule. In some instances, the small molecule is an inhibitor of the disease-associated molecule.

In some instances, the drug molecule comprises an oligonucleotide. In some instances, oligonucleotide comprises a double-stranded RNAi molecule or a single-stranded antisense oligonucleotide. In specific instances, the drug molecule comprises a small interfering RNA (siRNA), a microRNA (miRNA), an inhibitory double stranded RNA (dsRNA), a small or short hairpin RNA (shRNA), a piwi-interacting RNA (piRNA), a heterogeneous nuclear RNA (hnRNA), a small nuclear RNA (snRNA), or an enzymatically-prepared siRNA (esiRNA) or the precursors thereof. In other instances, the drug molecule comprises a nucleic acid disclosed in Sandy et al., Mammalian RNAi: a practical guide, BioTechniques, VOL. 39, NO. 2 REVIEW, which is incorporated by reference herein in its entirety.

In some instances, the gene expression construct encodes a gene editing enzyme. In some cases, the drug molecule comprises a CRISPR-based tool, a meganuclease-based tool, a zinc finger nuclease (ZFN)-based tool, or a transcription activator-like effector-based nuclease (TALEN)-based tool.

In some instances, the double-stranded RNAi molecule or the single-stranded antisense oligonucleotide is from about 9 to about 40 nucleotides in length. In some instances, the double-stranded RNAi molecule or the single-stranded antisense oligonucleotide is from about 9 to about 36 nucleotides in length. In some instances, the double-stranded RNAi molecule or the single-stranded antisense oligonucleotide is from about 10 to about 30 nucleotides in length. In some instances, the double-stranded RNAi molecule or the single-stranded antisense oligonucleotide is from about 15 to about 30 nucleotides in length. In some instances, the double-stranded RNAi molecule or the single-stranded antisense oligonucleotide is from about 20 to about 30 nucleotides in length. In some instances, the double-stranded RNAi molecule or the single-stranded antisense oligonucleotide is from about 10 to about 25 nucleotides in length. In some instances, the double-stranded RNAi molecule or the single-stranded antisense oligonucleotide is from about 10 to about 20 nucleotides in length. In some instances, the double-stranded RNAi molecule or the single-stranded antisense oligonucleotide is at least 5, 6, 7, 8, 9, 10, 15, 20, 25, or 30 nucleotides in length. In some instances, the double-stranded RNAi molecule or the single-stranded antisense oligonucleotide is no greater than 10, 15, 20, 25, 30, 35, 40, 45, 50, 55, 60, 65, or 70 nucleotides in length. In some instances, the double-stranded RNAi molecule or the single-stranded antisense oligonucleotide is about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In some instances, the drug molecule disclosed herein is a double-stranded RNAi molecule with a sense strand and an anti-sense strand. In some aspects, the sense strand and the antisense strand described herein are reverse complementary to each other and form a duplex with a 3′ overhang on the antisense strand. In some aspects, the sense strand and the antisense strand described herein are reverse complementary to each other and form a duplex with a 5′ overhang on the antisense strand. In some aspects, the sense strand and the antisense strand described herein are reverse complementary to each other and form a duplex with a 3′ overhang on the sense strand. In some aspects, the sense strand and the antisense strand described herein are reverse complementary to each other and form a duplex with a 5′ overhang on the sense strand.

In some instances, the double-stranded RNAi molecule or a single-stranded antisense oligonucleotide comprises at least one modified 2′ modified nucleotide. In some instances, the double-stranded RNAi molecule or a single-stranded antisense oligonucleotide comprises at least one modified internucleotide linkage.

In some instances, the double-stranded RNAi molecule or a single-stranded antisense oligonucleotide described herein comprises one or more sugar-modified nucleotide. In some specific instances, the sugar-modified nucleotide is a 2′-fluoro modified nucleotide. In some specific instances, the sugar-modified nucleotide is a 2′-alkoxy modified nucleotide (e.g., 2′-methoxy modified nucleotide). In some specific instances, the sugar-modified nucleotide is a 2′-amino modified nucleotide. In some specific instances, the sugar-modified nucleotide is a 2′-azido modified nucleotide.

In some instances, the double-stranded RNAi molecule or a single-stranded antisense oligonucleotide described herein comprises one or more backbone-modified nucleotide. In some specific instances, the modified backbone is a methylphosphonate. In some specific instances, the modified backbone is phosphorothioate. In some specific instances, the modified backbone is a guanidinopropyl phosphoramidate. In some specific instances, the modified backbone is a mesyl-phosphoramidate (MsPA) linkages. In some specific instances, the modified backbone is phosphorothioate, and the phosphorothioate is a stereochemically enriched phosphorothioate.

In some instances, the double-stranded RNAi molecule or a single-stranded antisense oligonucleotide described herein comprises one or more purine modification. In some specific instances, the purine modification described herein is 2,6-diaminopurine. In some specific instances, the purine modification described herein is 3-deaza-adenine. In some specific instances, the purine modification described herein is 7-deaza-guanine. In some specific instances, the purine modification described herein is 8-azido-adenine.

In some instances, the double-stranded RNAi molecule or a single-stranded antisense oligonucleotide described herein comprises one or more pyrimidine modification. In some specific instances, the pyrimidine modification described herein is 2-thio-thymidine. In some specific instances, the pyrimidine modification described herein is 5-carboxamide-uracil. In some specific instances, the pyrimidine modification described herein is 5-methyl-cytosine. In some specific instances, the pyrimidine modification described herein is 5-ethynyl uracil.

In some instances, the polynucleic acid molecule described herein comprises an abasic substitution. In those cases where a hybridized polynucleotide construct is contemplated for use as siRNA, a reduction of miRNA-like off-target effects is desirable. The inclusion of one or more (e.g., one or two) abasic substitutions in the hybridized polynucleotide constructs may reduce or even eliminate miRNA-like off-target effects, as the abasic substitutions lack nucleobases that are capable of engaging in base-pairing interactions and alleviate steric hindrance. Thus, the double-stranded RNAi molecule or a single-stranded antisense oligonucleotide disclosed herein may include one or more (e.g., one or two) abasic substitutions. In specific instances, abasic substitution is at the 5th nucleotide from the 5′ end of the antisense strand described herein. The polynucleotide molecule described herein may contain a strand including a seed region including a hypoxanthine nucleobase-containing nucleoside (e.g., inosine).

In some instances, the double-stranded RNAi molecule or a single-stranded antisense oligonucleotide described herein comprises one or more type of modifications as described above. Accordingly, in some instances, about 10% of the nucleotides from the double-stranded RNAi molecule or a single-stranded antisense oligonucleotide described herein are modified with one or more type of modifications as described above. In other instances, about 20% of the nucleotides from the double-stranded RNAi molecule or a single-stranded antisense oligonucleotide described herein are modified with one or more type of modifications as described above. In other instances, about 30% of the nucleotides from the double-stranded RNAi molecule or a single-stranded antisense oligonucleotide described herein are modified with one or more type of modifications as described above. In other instances, about 40% of the nucleotides from the double-stranded RNAi molecule or a single-stranded antisense oligonucleotide described herein are modified with one or more type of modifications as described above. In other instances, about 50% of the nucleotides from the double-stranded RNAi molecule or a single-stranded antisense oligonucleotide described herein are modified with one or more type of modifications as described above. In other instances, about 60% of the nucleotides from the double-stranded RNAi molecule or a single-stranded antisense oligonucleotide described herein are modified with one or more type of modifications as described above. In other instances, about 70% of the nucleotides from the double-stranded RNAi molecule or a single-stranded antisense oligonucleotide described herein are modified with one or more type of modifications as described above. In other instances, about 80% of the nucleotides from the double-stranded RNAi molecule or a single-stranded antisense oligonucleotide described herein are modified with one or more type of modifications as described above. In other instances, about 90% of the nucleotides from the double-stranded RNAi molecule or a single-stranded antisense oligonucleotide described herein are modified with one or more type of modifications as described above. In other instances, 100% of the nucleotides from the double-stranded RNAi molecule or a single-stranded antisense oligonucleotide described herein are modified with one or more type of modifications as described above.

In some instances, the one or more types of modifications described herein occurs at different positions within the double-stranded RNAi molecule or a single-stranded antisense oligonucleotide described herein. In specific instances, the one or more types of modifications described herein occurs in the seed region within the double-stranded RNAi molecule or a single-stranded antisense oligonucleotide described herein. In specific instances, the one or more types of modifications described herein occurs at 3′ terminal of the double-stranded RNAi molecule or a single-stranded antisense oligonucleotide described herein. In specific instances, the one or more types of modifications described herein occurs at 5′ terminal of the double-stranded RNAi molecule or a single-stranded antisense oligonucleotide described herein. In specific instances, the one or more types of modifications described herein occurs dispersedly within the double-stranded RNAi molecule or a single-stranded antisense oligonucleotide described herein. In specific instances, the one or more types of modifications described herein occurs in clusters within the double-stranded RNAi molecule or a single-stranded antisense oligonucleotide described herein.

In some instances, the disease-associated molecule is peripheral myelin protein 22 (PMP22). Accordingly, the drug molecule disclosed herein modifies PMP22. In some specific instances, PMP22 is associated with Charcot-Marie-Tooth Disease Type 1A (CMT1A). CMT damages the peripheral nerves system, and different types of CMT result in impaired electrical messaging to the muscles and their subsequent malfunctioning. Specifically, CMT1A, the most prevalent type of CMT (55%-60% of total CMT patient), usually begins during adolescence with muscle weakness and atrophy of the lower legs, followed by hand weakness and decreased sensations (e.g., touch, heat, cold) later in life. The disease is a progressive disease, whose symptoms usually worsen after the age of 45. Worsen symptoms comprise decline in the ability to perform simple tasks, instability in walking and tendency for injuries due to numerous falls, and reduced quality of life. The genetic defect in most CMT1A patients is a 1.5 Mb duplication on a chromosome segment containing the PMP22 gene. Elevation of PMP22 protein beyond basal concentration causes demyelization.

In some cases, the double-stranded RNAi molecule or the single-stranded antisense oligonucleotide comprises a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% complementary to a target region of human PMP22 transcript. In other cases, the double-stranded RNAi molecule or the single-stranded antisense oligonucleotide comprises a consecutive sequence of at least 10, 11, 12, 13, 14, 15, 16, 17, 18, 19, 20, 21, 22, 23, 24, or 25 nucleotides in length that is complementary to a target region of human PMP22 transcript with no more than 1, 2, 3, 4, or 5 mismatches. In other cases, the double-stranded RNAi molecule or the single-stranded antisense oligonucleotide comprises a consecutive sequence of about 10, 11, 12, 13, 14, 15, 16, 17, 18, 19, 20, 21, 22, 23, 24, or 25 nucleotides in length that is complementary to a target region of human PMP22 transcript with no more than 1, 2, 3, 4, or 5 mismatches. In some instances, the target region of human PMP22 transcript is in the 5′ UTR of human PMP22 transcript. In some instances, the target region of human PMP22 transcript is in the 3′ UTR of human PMP22 transcript. In some instances, the target region of human PMP22 transcript is in the coding region of human PMP22 transcript. In some instances, the target region of human PMP22 transcript is in exon 1 of human PMP22 transcript. In some instances, the target region of human PMP22 transcript is in exon 2 of human PMP22 transcript. In some instances, the target region of human PMP22 transcript is in exon 3 of human PMP22 transcript. In some instances, the target region of human PMP22 transcript is in exon 4 of human PMP22 transcript. In some instances, the target region of human PMP22 transcript is in exon 5 of human PMP22 transcript. In some instances, the target region of human PMP22 transcript is in exon 6 of human PMP22 transcript. In some instances, the target region of human PMP22 transcript is in exon 7 of human PMP22 transcript. In some instances, the target region of human PMP22 transcript comprises an intron portion of the pre-PMP22 transcript. In some instances, the target region of human PMP22 transcript comprises an exon-intron junction of the pre-PMP22 transcript.

In some instances, the disease-associated molecule is antiganglioside antibodies. Accordingly, the drug molecule disclosed herein modifies antiganglioside antibodies. In some cases, antiganglioside antibodies are associated with the Guillain-Barré syndrome. The Guillain-Barré syndrome is a post-infectious immune-mediated neuropathy, which results from the autoimmune destruction of nerves in the peripheral nervous system. The syndrome may include muscle movement as well as those that transmit pain, temperature and touch sensations. This can result in muscle weakness and loss of sensation in the legs and/or arms. Antiganglioside antibodies may be involved in the pathogenesis of the Guillain-Barré syndrome.

In some instances, the disease-associated molecule is a subunit of SWI/SNF protein complexes, which is encoded by gene SMARCB1. In some instances, the disease-associated molecule is LZTR1 protein, which is encoded by LZTR1 gene. Accordingly, the drug molecule disclosed herein modifies the subunit of SWI/SNF protein complexes. Accordingly, the drug molecule disclosed herein modifies LZTR1 protein. In some cases, the subunit of SWI/SNF protein complexes is associated with schwannomatosis. In some cases, LZTR1 protein is associated with schwannomatosis. Schwannomatosis is a type of neurofibromatosis that causes multiple nerve sheath tumors.

In some instances, the drug molecule is immunoglobulin, and the associated disease is chronic inflammatory demyelinating polyneuropathy (CIDP). CIDP is a rare disorder of the peripheral nerves characterized by gradually increasing symmetrical motor and sensory loss and weakness associated with loss of deep tendon reflexes. It is caused by damage to Schwann cells. The gradual onset of CIDP can delay diagnosis by several months or even years, resulting in significant nerve damage that may limit and delay the response to therapy. Most individuals may require long term treatment; nearly a third of CIDP patients may progress to wheelchair dependence if left untreated. Early recognition and proper treatment are critical in helping patients avoid a significant amount of disability.

### Coupling Target Moieties and Drug Molecules

In certain instances, the targeting moieties disclosed herein can be coupled or conjugated to the drug molecules disclosed herein directly (e.g., through a covalent or non-covalent linkage), or through a linker (e.g., a cleavable linker versus a non-cleavable linker, or a peptide linker versus a non-peptide linker), or through an amino acid or other functional group.

In some instances, the targeting moieties disclosed herein and the drug molecules disclosed herein are connected via a non-covalent interaction. In specific cases, the non-covalent interaction comprises a biotin/avidin interaction. Examples of connecting connect the targeting moiety and the drug molecule include, but are not limited to, the use of biotin and avidin or streptavidin (see, e.g., U.S. Pat. No. 4,885,172 A), typical biotin/avidin alternatives (e.g., FITC/anti-FITC (see, e.g., Harmer and Samuel (1989) J. Immunol. Meth. 122 (1): 115-221), dioxigenin/anti-dioxigenin, and the like.

In other instances, the targeting moieties disclosed herein and the drug molecules disclosed herein are connected via a covalent interaction. In specific cases, the targeting moieties disclosed herein and the drug molecules disclosed herein are connected via chemical conjugation. Traditional chemical conjugation using, for example, bifunctional coupling agents such as glutaraldehyde, diimide esters, aromatic and aliphatic diisocyanates, bis-p-nitrophenyl esters of dicarboxylic acids, aromatic disulfonyl chlorides and bifunctional arylhalides such as 1,5-difluoro-2,4-dinitrobenzene; p,p′-difluoro m,m′-dinitrodiphenyl sulfone, sulfhydryl-reactive maleimides, and the like.

In some cases, the chemical conjugation of the targeting moieties disclosed herein and the drug molecules disclosed herein is via a non-cleavable linker or via a cleavable linker.

In some instances, the cleavable linkers are generally cleavable only intracellularly and are preferably stable in extracellular environments, e.g. extracellular to the Schwann cells. In specific cases, the cleavable linker disclosed herein comprises a chemically cleavable linkers. In specific cases, the cleavable linker disclosed herein comprises an enzymatically cleavable linker.

A number of different chemically cleavable linkers are known to those of skill in the art (see, e.g., U.S. Pat. Nos. 4,618,492; 4,542,225, and 4,625,014). In some specific instances, the chemically cleavable linker is pH-sensitive linker. In some cases, the pH-sensitive linker disclosed herein is readily degrades in high pH environments. In some cases, the pH-sensitive linker disclosed herein is readily degrades in low pH environments. In some instances, the pH-sensitive linker may be cleaved at a pH in a range of 4 to 6. In some instances, the pH-sensitive linker comprises a hydrazone or cyclic acetal. In some instances, the pH-sensitive linker is cleaved within an endosome or a lysosome. In other specific instances, the chemically cleavable linker is glutathione-sensitive linker. In some cases, the glutathione-sensitive linker is cleaved by an disulfide exchange reaction with a glutathione species inside a cell. In some instances, the disulfide moiety further comprises at least one amino acid, e.g. a cysteine residue. Other illustrative chemically cleavable linkers include, but are not limited to, acid-labile linkers, disulfide linkers, and the like. Acid-labile linkers are designed to be stable at pH levels encountered in the blood, but become unstable and degrade when the low pH environment in lysosomes is encountered. an acid-labile linker comprising a moiety selected from the group consisting of a hydrazone, an acetal, a cis-aconitate amide, and a silyl ether. Acid-sensitive linkers include, but are not limited to hydrazones, acetals, cis-aconitate-like amides, and silyl ethers (see, e.g., Perez et al. (2013) Drug Discov. Today, 1-13). Hydrazones are easily synthesized and have a plasma half-life of 183 hours at pH 7 and 4.4 hours at pH 5, indicating that they are selectively cleavable under acidic conditions such as those found in the lysosome (see, e.g., Doronina et al. 92013) Nat. Biotechnol. 21 (7): 778-784). Disulfide bridges are cleavable linkers that take advantage of the cellular reducing environment (see, e.g., Saito et al. (2013) Adv. Drug Deliv. Rev. 55 (2): 199-215). After internalization and degradation, disulfide bridges can release drugs in the lysosome.

Enzymatically cleavable linkers are selected to be cleaved by an enzyme (e.g., a protease). In some instances, protease-cleavable linkers are typically designed to be stable in blood/plasma, but are rapidly cleaved in lysosomes by lysosomal enzymes. The most popular enzymatic cleavage sequence is the dipeptide valine-citrulline, combined with a self-immolative linker p-aminobenzyl alcohol (PAB). Cleavage of an amide-linked PAB triggers a 1,6-elimination of carbon dioxide and concomitant release of the free drug in parent amine form (see, e.g., Burke et al. (2009) Bioconjug. Chem. 20 (6): 1242-1250). In some specific instances, the cleavable linker is a protease-sensitive linker. The protease-sensitive linker described herein may comprise a sequence cleavable by a lysosomal protease and/or an endosomal protease. In some cases, the protease-sensitive linker typically comprises peptide sequences and may be 2-10 amino acids, about 2-5 amino acids, about 5-10 amino acids, about 10 amino acids, about 5 amino acids, about 3 amino acids, or about 2 amino acids in length. In some instances, the protease-sensitive linker comprises naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids. In some instances, the protease-sensitive linker comprises a valine-citrulline or alanine-citrulline dipeptide sequence. In some instances, the protease-sensitive linker can be cleaved by a lysosomal protease, e.g. cathepsin B, and/or an endosomal protease.

In some instances, the targeting moiety and the drug molecule are covalently linked to each other via a non-cleavable linker. Generally, the non-cleavable linker cannot be readily degraded in a cellular or physiological environment. In some instances, the non-cleavable linker described herein comprises an optionally substituted alkyl group, wherein the substitutions may include halogens, hydroxyl groups, oxygen species, and other common substitutions. In some instances, the non-cleavable linker described herein comprises an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some instances, sortase-mediated ligation is utilized to covalently link the drug molecule comprising a LPXT sequence to the targeting moiety comprising a (G). In other instances, sortase-mediated ligation is utilized to covalently link the targeting moiety comprising a LPXT sequence to the drug molecule comprising a (G) sequence (see, e.g. Proft T. Sortase-mediated protein ligation: an emerging biotechnology tool for protein modification and immobilization. Biotechnol Lett. 2010, 32 (1): 1-10, which is incorporated by reference herein in its entirety). In some instances, the non-cleavable linkers described herein comprises a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S; an imino, an optionally substituted nitrogen species, an optionally substituted oxygen species O, an optionally substituted sulfur species, or a poly(alkylene oxide), e.g. polyethylene oxide or polypropylene oxide.

In some cases, the linkers described herein are peptide linkers. In various instances, the peptide linker is relatively short, typically about 20 amino acids or less, about 15 amino acids or less, about 10 amino acids or less, about 8 amino acids or less, about 5 amino acids or less, about 3 amino acids or less, or is a single amino acid. In some cases, the peptide linker disclosed herein comprises an amino acid sequence cleavable by a protease. In some cases, the peptide linker disclosed herein comprises an amino acid sequence cleavable by a cathepsin. In some cases, the peptide linker disclosed herein comprises a dipeptide valine-citrulline (Val-Cit) (see U.S. Pat. No. 6,214,345, incorporated herein by reference in its entirety) or Phe-Lys. A library of dipeptide linkers was screened by Debowchik and co-workers to measure the rate of doxorubicin release by enzymatic hydrolysis (see, e.g., Dubowchik et al. (2002) Bioconjug. Chem. 13 (4): 855-869; Dubowchik et al. (2002) Bioorg. Med. Chem. Lett. 12 (11): 1529-1532). They found that Phe-Lys was cleaved most rapidly with a half-life of 8 min, followed closely by Val-Lys with a half-life of 9 min. In stark contrast, Val-Cit showed a half-life of 240 min. They also found that removal of the PAB group reduced the cleavage rate, presumably through steric interference with enzyme binding. Another study compared the potency of auristatin derivative MMAE linked by dipeptide linkers Phe-Lys and Val-Cit and an analogous hydrazone linker. The Val-Cit linker proved to be over 100 times as stable as the hydrazone linker in human plasma. Most significantly, the Phe-Lys linker was substantially less stable than Val-Cit in human plasma, which accounts for its current popularity (see, e.g., Doronina et al. (2003) Nat. Biotechnol. 21 (7): 778-784).

Other peptide linkers include, but not limited to, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, GGGG, PSGSP, PSPSP, KKKK, RRRR, ASASA, GGSGGS, GGGGS, GGGGS GGGGS, GGGGS GGGGS GGGGS, GGGGS GGGGS GGGGS GGGGS, GGGGS GGGGS GGGGS GGGGS GGGGS, GGGGS GGGGS GGGGS GGGGS GGGGS GGGGS, GGGGS GGGGS GGGGS FK GGGGS GGGGS GGGGS, and GGGGS GGGGS GGGGS VA GGGGS GGGGS GGGGS.

In some cases, the linkers described herein utilize PASYLATION®-technology, which is a genetic fusion with conformationally disordered polypeptide sequences composed of the amino acids Pro, Ala, and/or Ser. In other cases, the linkers described herein are selected from the ones disclosed in patents such as U.S. Pat. No. 10,961,287, which is incorporated by reference herein in its entirety.

In some cases, the linkers described herein are non-peptide linkers. A glucuronide linker incorporates a hydrophilic sugar group that is cleaved by the lysosomal enzyme beta glucuronidase. Once the sugar is cleaved from the phenolic backbone, self-immolation of the PAB group releases the conjugated moiety (see, e.g., Jeffrey et al. (2006) Bioconjug. Chem. 17 (3): 831-840). In some cases, the linkers described herein are derived from natural multi-domain proteins (see, e.g., Chen at al., (2013) Adv Drug Deliv Rev. 65 (10): 1357-1369).

The targeting moieties disclosed herein may be attached to the drug molecules disclosed herein via certain functional groups or via certain reactions. In some cases, the linker disclosed herein comprises a functional group that is reactive with a corresponding functional group on the targeting moieties disclosed herein and/or the drug molecules disclosed herein. A bifunctional linker has one functional group reactive with a group on the targeting moieties disclosed herein and another functional group reactive on the drug molecules disclosed herein and can be used to form the desired conjugate. In some instances, a heterobifunctional linker comprises two or more different reactive groups that react with sites on the targeting moieties disclosed herein and the drug molecules disclosed herein, respectively. For example, a heterobifunctional crosslinker such as cysteine may comprise an amine reactive group and a thiol-reactive group can interact with an aldehyde on a derivatized peptide. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. Such reactions and functional groups are illustrative and non-limiting. Other illustrative suitable reactive groups include, but are not limited to thiol (—SH), carboxylate (COOH), carboxyl (—COOH), carbonyl, amine (NH2), hydroxyl (—OH), aldehyde (—CHO), alcohol (ROH), ketone (R2CO), active hydrogen, ester, sulfhydryl (SH), phosphate (—PO3), or photoreactive moieties. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

In some instances, the linkers described herein connect the targeting moiety to the drug molecule by a cycloaddition reaction between an azide and an alkyne to form a triazole, wherein the azide and the alkyne may be located on the targeting moiety, the drug molecule, or the linker. In some instances, an alkyne may be a cyclic alkyne, e.g., a cyclooctyne. In some instances, an alkyne may be bicyclononyne (also known as bicyclo[6.1.0]nonyne or BCN) or substituted bicyclononyne. In some instances, a cyclooctane is as described in International Patent Application Publication WO2011136645, published on Nov. 3, 2011, entitled, “Fused Cyclooctyne Compounds And Their Use In Metal free Click Reactions”. In some instances, an azide may be a sugar or carbohydrate molecule that comprises an azide. In some instances, an azide may be 6-azido-6-deoxygalactose or 6-azido-N-acetylgalactosamine. In some instances, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some instances, a cycloaddition reaction between an azide and an alkyne to form a triazole, wherein the azide and the alkyne may be located on the targeting moiety, drug molecule, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosarninyltransferase”.

In some instances, the linkers described herein connect to the targeting moiety to the drug molecule by the Diels-Alder reaction between a dienophile and a diene/hetero-diene, wherein the dienophile and the diene/hetero-diene may be located on the targeting moiety, the drug molecule, or the linker. In some instances a linker is connected to the targeting moiety and/or the drug molecule by other pericyclic reactions, e.g. ene reaction. In some instances, the linker disclosed herein is connected to the targeting moiety and/or the drug molecule by an amide, thioamide, or sulfonamide bond reaction. In some instances, the linker disclosed herein connect to the targeting moiety to the drug molecule by a condensation reaction to form an oxime, hydrazone, or semicarbazide group existing between the linker, the targeting moiety, and the drug molecule.

In some instances, the linker disclosed herein connect the targeting moiety to the drug molecule by a conjugate addition reactions between a nucleophile, e.g. an amine or a hydroxyl group, and an electrophile, e.g. a carboxylic acid or an aldehyde. In some instances, a nucleophile may exist on a linker and an electrophile may exist on the targeting moiety or the drug molecule prior to a reaction between a linker and the targeting moiety or the drug molecule. In some instances, an electrophile may exist on a linker and a nucleophile may exist on the targeting moiety or the drug molecule prior to a reaction between a linker and the targeting moiety or the drug molecule. In some instances, an electrophile may be an azide, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some instances, a nucleophile may be an optionally substituted alkene, an optionally substituted alkyne, an optionally substituted aryl, an optionally substituted heterocyclyl, a hydroxyl group, an amino group, an alkylamino group, an anilido group, or a thiol group.

In some instances, the linkers described herein further comprises a spacer, e.g., a polyethylene glycol spacer or an acyl/carbomoyl sulfamide spacer, e.g., a HydraSpace™ spacer. In some instances, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12, which is incorporated by reference herein in its entirety.

Generally the linkers disclosed herein have no specific biological activity other than to join the proteins or to preserve some minimum distance or other spatial relationship between them. However, the constituent amino acids of the spacer may be selected to influence some property of the molecule such as the folding, net charge, or hydrophobicity. In certain instances the linker may comprise an enzymatic cleavage site.

After chemical synthesis, biological expression, or purification, the fusion protein may possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (See, Debinski et al. (1993) J. Biol. Chem., 268:14065-14070; Kreitman and Pastan (1993) Bioconjug. Chem., 4:581-585; and Buchner, et al. (1992) Anal. Biochem., 205:263-270).

In some instances, one of the targeting moiety and drug molecule is a peptide/protein or an antibody or an antigen binding fragment thereof and the other is a nucleotide. In some cases, the peptide/protein or an antibody or an antigen binding fragment thereof comprises a non-natural amino acid to which the nucleotides can be covalently linked. In some cases, the peptide/protein or an antibody or an antigen binding fragment thereof is covalently linked to the nucleotide via conjugation to a lysine residue or a cysteine residue of the peptide/protein or an antibody or an antigen binding fragment thereof. In some cases, the nucleotide is conjugated to a cysteine residue of the peptide/protein or an antibody or an antigen binding fragment thereof via a maleimide-containing linker, optionally wherein the maleimide-containing linker comprises a maleimidocaproyl or maleimidomethyl cyclohexane-1-carboxylate group. In some instances, peptide/protein or an antibody or an antigen binding fragment thereof is glycosylated that comprises at least one sugar moiety to which the nucleotide is covalently linked. In some cases, the at least one sugar moiety comprises at least one sugar moiety that is a branched mannose. In some cases, the peptide/protein or an antibody or an antigen binding fragment thereof is glycosylated that comprises more than one sugar moieties, and each of all or a portion of the more than one sugar moieties is covalently linked to a separate nucleotide. In some cases, the peptide/protein or an antibody or an antigen binding fragment thereof is a fully-glycosylated or a partially-glycosylated. A partially-glycosylation may be produced via chemical or enzymatic means. In some cases, a partially-glycosylation is produced in a cell that is deficient for an enzyme in the N- or O-glycosylation pathway. In some instances, the linkers described herein are connected to the peptide/protein or an antibody or an antigen binding fragment thereof via a phosphate, thioether, ether, carbon-carbon, or amide bond. In some instances, the linkers described herein are connected to the nucleotide through a phosphate or phosphorothioate group, e.g. a terminal phosphate of an oligonucleotide backbone.

In some specific cases, one of the targeting moiety and drug molecule is a peptide/protein or an antibody or an antigen binding fragment thereof and the other is a double-stranded RNAi molecule. Accordingly, in some instances, the targeting moiety described herein is conjugated to 3′ end of the sense strand. In some instances, the targeting moiety described herein is conjugated to 5′ end of the sense strand. In some instances, the targeting moiety described herein is conjugated to 3′ end of the antisense strand. In some instances, the targeting moiety described herein is conjugated to 5′ end of the antisense strand.

In some instances, both the targeting moiety and drug molecule are peptides/proteins or antibodies or antigen binding fragments thereof. Accordingly, in some cases, the targeting moiety and drug molecule can be expressed as a fusion protein. In some specific cases, the targeting moiety and drug molecule are directly attached to each other. In some specific cases, the targeting moiety and drug molecule are connected via an intervening amino acid. In some specific cases, the targeting moiety and drug molecule are connected through a peptide linker. Specifically, the linkers can be joined to the constituent amino acids through their side groups (e.g., through a disulfide linkage to cysteine), while in other instances, the linkers are joined to the alpha carbon amino and carboxyl groups of the terminal amino acids when such are present.

For generating the fusion protein, generally this involves creating a DNA sequence that encodes the fusion protein, placing the DNA in an expression cassette under the control of a particular promoter, expressing the protein in a host, isolating the expressed protein and, if required, renaturing the protein. DNA encoding the fusion proteins can be prepared by any suitable method, including, for example, cloning and restriction of appropriate sequences or direct chemical synthesis by methods such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetra. Lett., 22:1859-1862; and the solid support method of U.S. Pat. No. 4,458,066. In certain instances, DNA encoding fusion proteins constructs described herein may be cloned using DNA amplification methods such as polymerase chain reaction (PCR). Thus, for example, the nucleic acid sequence encoding the targeting moieties disclosed herein can be PCR amplified, using primers containing the designed restriction sites. This produces a nucleic acid encoding the targeting moieties disclosed herein and having terminal restriction sites. Similarly the nucleic acid encoding the drug molecules disclosed herein can be provided having complementary restriction sites. Ligation of sequences and insertion into a vector produces a vector encoding the fusion protein. The nucleic acid sequences encoding the fusion proteins can be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences. The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant fusion proteins can be purified according to standard procedures of the art, including ammonium sulfate precipitation, affinity columns, column chromatography, gel electrophoresis and the like (see, generally, R. Scopes (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically.

In some cases, certain modifications can be made to the fusion proteins without diminishing their biological activity. Some modifications may be made to facilitate the cloning, expression, or incorporation of the drug molecules or targeting moieties disclosed herein into a fusion protein. Such modifications include, for example, a methionine added at the amino terminus to provide an initiation site, or additional amino acids placed on either terminus to create conveniently located restriction sites or termination codons.

In some instances, one of the targeting moiety and drug molecule is a peptide/protein or an antibody or an antigen binding fragment thereof and the other is a small molecule. The exemplary linkers can be found in U.S. Pat. Nos. 10,864,279, 9,814,784, 11173214, 11104968, 9872924, 10537644, U.S. patent applications US20180169262, US20220072137, and US20180311375, which are incorporated by reference herein in their entireties.

In some instances, one of the targeting moiety and drug molecule is a double-stranded RNAi molecule or a single-stranded antisense oligonucleotide and the other is a small molecule. Accordingly, the exemplary linkers can be found in U.S. Pat. Nos. 8,772,472, 9,211,343, 8,969,543, 9,074,208, 10947323, U.S. patent applications US20120177723, US20120108803, US20040053876, US20210108200, and US20150315587, which are incorporated by reference herein in their entireties.

### Other Properties of Drug Conjugates and Pharmaceutical Compositions

In certain instances, multiple targeting moieties disclosed herein can be attached to a single drug molecule disclosed herein. In certain instances, multiple the drug molecules disclosed herein can be attached to a single targeting moiety disclosed herein. In certain instances a single targeting moiety disclosed herein is attached to a single the drug molecule disclosed herein. In some cases, the drug molecule to the targeting moiety ratio is 1:1. In some cases, the drug molecule to the targeting moiety ratio is about 2:1, 3:1, 4:1, 5:1, 6:1, 7:1, 8:1, 9:1, or 10:1. In some cases, the drug molecule to the targeting moiety ratio is about 1:2, 1:3, 1:4, 1:5, 1:6, 1:7, 1:8, 1:9, or 1:10.

In some aspects, provided herein are pharmaceutical compositions comprising drug conjugates disclosed herein and pharmaceutically acceptable excipients. In some instances, the pharmaceutical compositions are formulated for parenteral, intravenous, subcutaneous, intrathecal, or intrasciatic injection.

In some instances, the drug conjugate provided herein preferably internalizes to Schwann cells. In some instances, the drug conjugate provided herein internalizes to Schwann cells at least 1, 2, 3, 4, 5, 6, 7, 8, 9, or 10 fold(s) more than it internalizes to a cell type other than Schwann cells.

In some instances, the drug conjugate provided herein enters blood stream and stays intact. In some instances, the drug conjugate provided herein enters blood stream and effectively crosses the blood nerve barrier. In some instances, the drug conjugate provided herein enters blood stream, effectively crosses the blood nerve barrier, and enriches in sciatic nerves.

The pharmaceutical composition described herein can be prepared to include the drug conjugates disclosed herein, in a form suitable for administration to a subject using carriers, excipients, and vehicles. Exemplary excipients include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable vehicles include aqueous solutions, non-toxic excipients, including salts, preservatives, buffers and the like, as described, for instance, in Remington: The Science and Practice of Pharmacy, 21st Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions described herein may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, intrasciatic, intrathecal, and the like), oral administration, ophthalmic application, inhalation, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. In some instances, the composition will typically be sterile and fluid to the extent that easy syringability exists. In some instances, the composition will be stable under the conditions of manufacture and storage and preserved against the contaminating action of microorganisms, such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

In some cases, the pharmaceutical composition is formulated in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the required pharmaceutical vehicle. The specification for the dosage unit forms are related to the characteristics of the pharmaceutical composition and the particular therapeutic effect to be achieve. The principal pharmaceutical composition is compounded for convenient and effective administration in effective amounts with a suitable pharmaceutically acceptable vehicle in an acceptable dosage unit. In the case of compositions containing supplementary active ingredients, the dosages are determined by reference to the usual dose and manner of administration of the ingredients.

The pharmaceutical composition can be orally administered, for example, in a carrier, e.g., in an enteric-coated unit dosage form. The pharmaceutical composition and other ingredients can also be enclosed in a hard or soft-shell gelatin capsule or compressed into tablets. For oral therapeutic administration, the pharmaceutical composition can be incorporated with excipients and used in the form of ingestible tablets, troches, capsules, pills, wafers, and the like. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Any material used in preparing any dosage unit form should be of pharmaceutically acceptable purity and substantially non-toxic in the amounts employed. In addition, the pharmaceutical composition can be incorporated into sustained-release preparations and formulations.

For the polynucleotide molecule described herein, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

In some cases, the drug molecules disclosed herein are encapsulated within a nanoparticulate system which comprises a plurality of particles, such as but are not limited to, polymers, gold particles, silicas, lipids, or protein-base particles, and the plurality of particles are formulated to incorporate with the targeting moieties disclosed herein (see WO 2023/056282, incorporated by reference herein in its entirety). In some cases, the plurality of particles are coupled with the targeting moieties disclosed herein by non-covalent binding (e.g., absorption), and such coupling maintains the biological activities of the targeting moieties. In some cases, the plurality of particles are coupled with the targeting moieties disclosed herein by covalent binding, and such coupling maintains the biological activities of the targeting moieties. In some cases, the absorption is a non-covalent immobilization strategy, such as but is not limited to, physical absorption or ionic binding. In some cases, the physical absorption comprises an attachment of the targeting moieties disclosed herein to the particles' surface through weak interactions (e.g., electrostatic, hydrogen binding, hydrophobic, or van der Waals attractive forces). In some cases, the ionic binding is based on ionic linkages between oppositely charged surfaces of the targeting moieties disclosed herein and the particles. In some cases, the covalent binding requires prior activation of the particles. In some cases, the covalent binding is based on carbodiimide chemistry, maleimide chemistry (see Jahan S. T et al. Int. J. Drug Deliv. 2017, incorporated by reference herein in its entirety), or “click chemistry” (see Yi, G et al. Biomater. Res. 2018, incorporated by reference herein in its entirety). In some specific cases, the targeting moieties comprise the antibodies or antigen binding fragments thereof described herein, which bind to the nanoparticulate system using maleimide-thiol chemistry (see Niels Dammes et al. Nat Nanotechnol. 2021, incorporated by reference herein in its entirety). In some specific cases, the target moieties disclosed herein are conjugated on the surface of the nanoparticulate system comprising a plurality of polymeric nanoparticles (see Nasrul Wathoni, et al. Drug Delivery., 2022, incorporated by reference herein in its entirety).

In some instances, the drug molecules disclosed herein are encapsulated in an extracellular vesicle (EV), and the EV is modified to present the targeting moieties disclosed herein on its outside surface (see WO 2020/141369 or Chan., H., et al, Front. Cell. Dev. Biol., 2021, 9, 751079, incorporated by reference herein in its entirety). In some specific cases, the targeting moieties disclosed herein are tethered to the surface of the EV by fusing with a lysosome-associated membrane glycoprotein 2b protein (see Liang., Y et al., ACS Appl Mater Interfaces., 2020, 12, 36938, incorporated by reference herein in its entirety). In some specific cases, the targeting moieties disclosed herein are tethered to the surface of the EV via a bio-orthogonal chemistry (see Tian., T., et al., Biomaterials, 2018., 150, 137, incorporated by reference herein in its entirety).

### Methods

In some aspects, provided herein are methods of targeted delivery of drugs to Schwann cells, by contacting the drug conjugates disclosed herein or the pharmaceutical compositions comprising the drug conjugates disclosed herein to the Schwann cells. In some instances, the drug conjugates are internalized into the Schwann cells upon binding of the targeting moieties to the cell surface molecule expressed on the Schwann cells. In some instances, the cell surface molecule expressed on the Schwann cells is a cell surface receptor having an extracellular domain. In some instances, the cell surface molecules are enriched on the Schwann cell surface. In some instances, the drug conjugates are delivered to the Schwann cells by systemic delivery of the drug conjugates disclosed herein or the pharmaceutical compositions disclosed herein to the body of a subject. In some instances, the drug conjugates disclosed herein can penetrate the blood nerve barrier. In some instances, the drug conjugates discloses herein can penetrate through the blood nerve barrier and can be enriched in sciatic nerves. In some aspects, provided herein are methods of preferable delivery of drugs to or into the Schwann cells, by contacting the drug conjugates disclosed herein or the pharmaceutical compositions comprising the drugs disclosed herein to the Schwann cells. In some instances, the drug conjugates are preferably internalized into the Schwann cells upon binding of the targeting moieties to the cell surface molecule expressed on the Schwann cells. In some instances, the cell surface molecule preferably expressed on the Schwann cells is a cell surface receptor having an extracellular domain. In some instances, the cell surface molecules are preferably expressed on the Schwann cell surface. In some instances, the cell surface molecules are expressed at least 10%, 20%, 30%, 40%, 50%, 60%, 70%, 80%, 90%, or 2, 3, 4, 5 folds more in Schwann cells than other types of cells. In some instances, the methods provided herein ensure that the drugs are preferably internalized into the Schwann cells at least 10%, 20%, 30%, 40%, 50%, 60%, 70%, 80%, 90%, or 2, 3, 4, 5, 6, 7, 8, 9, or 10 fold(s) more than being internalized into a cell type other than Schwann cells. In some instances, the drug conjugates are delivered to the Schwann cells by systemic delivery of the drug conjugates disclosed herein or the pharmaceutical compositions disclosed herein to the body of a subject. In some instances, the drug conjugates disclosed herein can penetrate the blood nerve barrier. In some instances, the drug conjugates discloses herein can penetrate through the blood nerve barrier and can be enriched in sciatic nerves.

In some instances, the methods of targeted delivery disclosed herein possess improved pharmacokinetics. In specific instances, the methods of targeted delivery disclosed herein result in a higher Cmax (i.e., maximum concentration of the drug molecule in Schwann cells). In specific instances, the methods of targeted delivery disclosed herein result in a shorter Tmax when Cmax is reached in Schwann cells. In specific instances, the methods of targeted delivery disclosed herein result in a higher area under the concentration-time curve. In specific instances, the methods of targeted delivery disclosed herein result in a longer t_{1/2} (i.e., the time taken for the concentration of the drug molecule to fall by one half once distribution equilibrium has been reached in Schwann cells). Accordingly, in some cases, the methods of targeted delivery disclosed herein result in the circulation and availability of the drug conjugate in the subject for at least 1, 5, 10, 15, 20, 25, 30, 35, or 40 days. In some cases, the methods of targeted delivery disclosed herein result in the circulation and availability of the drug conjugate in the subject for about 10, 20, 30, 40, 50, 60, 70, 80, 90, 100, 110, 120 days. In some cases, the methods of targeted delivery disclosed herein result in the circulation and availability of the drug conjugate in the subject for at most 90, 120, 150, 180, 210, or 240 days.

In some instances, the methods of targeted delivery disclosed herein result in reduced distribution in any other tissues, organs, or cell types than Schwann cells. In some instances, the methods of targeted delivery disclosed herein result in an insignificant amount or a minimum amount in any other tissues, organs, or cell types than Schwann cells. In some instances, the methods of targeted delivery disclosed herein are non-immunogenic. In some instances, the methods of targeted delivery disclosed herein are biodegradable. In some instances, the methods of targeted delivery disclosed herein are applicable for a varieties of different drug molecules.

In some instances, the methods of targeted delivery are achieved by parenteral, intravenous, subcutaneous, intrathecal, or intrasciatic injection of the pharmaceutical compositions disclosed herein. In some instances, the methods of targeted delivery are achieved by intravenous injection of the pharmaceutical compositions disclosed herein. In some instances, the methods of targeted delivery are achieved by topical delivery (see, e.g., Pitiot et al. (2022) Antibodies (Basel) 11 (3): 56.)

In another aspect, provided herein are methods of treating or preventing a peripheral demyelinating disease or a peripheral neuropathy, or alleviating or reducing symptoms of the peripheral demyelinating disease or the peripheral neuropathy in a subject in need thereof, comprising: providing the drug conjugates disclosed herein or the pharmaceutical compositions disclosed herein; and administering the subject the drug conjugates or the pharmaceutical compositions to treat the peripheral demyelinating disease or the peripheral neuropathy, to alleviate or reduce symptoms of the peripheral demyelinating disease or the peripheral neuropathy.

In some instances, the peripheral demyelinating disease or the peripheral neuropathy is Charcot-Marie-Tooth disorder, Guillain-Barré syndrome (acute inflammatory demyelinating polyradiculopathy type), schwannomatosis, chronic inflammatory demyelinating polyneuropathy, nerve trauma, post-chemotherapy neuropathy, diabetic peripheral neuropathy, migraine, Schwannomas, neurofibromatosis type 1 (NF1), malignant peripheral nerve sheath tumors (MPNSTs), or nerve injury.

In some instances where an elevation of the disease-associated molecule leads to the disease, the drug conjugate or the pharmaceutical composition is administered in a dose and schedule effective to reduce expression of the disease-associated molecule by at least 5%, 10%, 15%, 20%, 25%, 30%, 35%, 40%, 45%, 50%, 55%, 60%, 65%, 70%, 75%, 80%, 85%, 90%, or 95%. In some instances where an elevation of the disease-associated molecule leads to the disease, the drug conjugate or the pharmaceutical composition is administered in a dose and schedule effective to reduce expression of the disease-associated molecule by about 5%, 10%, 15%, 20%, 25%, 30%, 35%, 40%, 45%, 50%, 55%, 60%, 65%, 70%, 75%, 80%, 85%, 90%, or 95%. In some instances where an elevation of the disease-associated molecule leads to the disease, the drug conjugate or the pharmaceutical composition is administered in a dose and schedule effective to reduce activity of the disease-associated molecule by at least 5%, 10%, 15%, 20%, 25%, 30%, 35%, 40%, 45%, 50%, 55%, 60%, 65%, 70%, 75%, 80%, 85%, 90%, or 95%. In some instances where an elevation of the disease-associated molecule leads to the disease, the drug conjugate or the pharmaceutical composition is administered in a dose and schedule effective to reduce activity of the disease-associated molecule by about 5%, 10%, 15%, 20%, 25%, 30%, 35%, 40%, 45%, 50%, 55%, 60%, 65%, 70%, 75%, 80%, 85%, 90%, or 95%.

In some instances, the drug conjugate or the pharmaceutical composition is administered in a dose and schedule effective to reduce the expression of PMP22 by at least 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the expression of PMP22 by about 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the activity of PMP22 by at least 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the activity of PMP22 by about 5%, 10%, 15%, 20%, 25%, 30%, 35%, 40%, 45%, 50%, 55%, 60%, 65%, 70%, 75%, 80%, 85%, 90%, or 95%.

In some instances, the drug conjugate or the pharmaceutical composition is administered in a dose and schedule effective to reduce the expression of antiganglioside antibodies by at least 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the expression of antiganglioside antibodies by about 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the activity of antiganglioside antibodies by at least 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the activity of antiganglioside antibodies by about 5%, 10%, 15%, 20%, 25%, 30%, 35%, 40%, 45%, 50%, 55%, 60%, 65%, 70%, 75%, 80%, 85%, 90%, or 95%.

In some instances where a lack of the disease-associated molecule leads to the disease, the drug conjugate or the pharmaceutical composition is administered in a dose and schedule effective to increase expression of the disease-associated molecule by at least 5%, 10%, 15%, 20%, 25%, 30%, 35%, 40%, 45%, 50%, 55%, 60%, 65%, 70%, 75%, 80%, 85%, 90%, or 95%. In some instances where a lack of the disease-associated molecule leads to the disease, the drug conjugate or the pharmaceutical composition is administered in a dose and schedule effective to increase expression of the disease-associated molecule by about 5%, 10%, 15%, 20%, 25%, 30%, 35%, 40%, 45%, 50%, 55%, 60%, 65%, 70%, 75%, 80%, 85%, 90%, or 95%. In some instances where a lack of the disease-associated molecule leads to the disease, the drug conjugate or the pharmaceutical composition is administered in a dose and schedule effective to increase activity of the disease-associated molecule by at least 5%, 10%, 15%, 20%, 25%, 30%, 35%, 40%, 45%, 50%, 55%, 60%, 65%, 70%, 75%, 80%, 85%, 90%, or 95%. In some instances where a lack of the disease-associated molecule leads to the disease, the drug conjugate or the pharmaceutical composition is administered in a dose and schedule effective to increase activity of the disease-associated molecule by about 5%, 10%, 15%, 20%, 25%, 30%, 35%, 40%, 45%, 50%, 55%, 60%, 65%, 70%, 75%, 80%, 85%, 90%, or 95%.

In some instances, the drug conjugate or the pharmaceutical composition is administered in a dose and schedule effective to increase the expression of the subunit of SWI/SNF protein complexes encoded by gene SMARCB1 by at least 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to increase the expression of the subunit of SWI/SNF protein complexes encoded by gene SMARCB1 by about 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the activity of the subunit of SWI/SNF protein complexes encoded by gene SMARCB1 by at least 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the activity of the subunit of SWI/SNF protein complexes encoded by gene SMARCB1 by about 5%, 10%, 15%, 20%, 25%, 30%, 35%, 40%, 45%, 50%, 55%, 60%, 65%, 70%, 75%, 80%, 85%, 90%, or 95%.

In some instances, the drug conjugate or the pharmaceutical composition is administered in a dose and schedule effective to increase the expression of LZTR1 protein encoded by gene LZTR1 by at least 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to increase the expression of LZTR1 protein encoded by gene LZTR1 by about 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the activity of LZTR1 protein encoded by gene LZTR1 by at least 5%, 10%, 15%, 20%, 25%, 30%, 35%, 40%, 45%, 50%, 55%, 60%, 65%, 70%, 75%, 80%, 85%, 90%, or 95%. In some instances, the drug conjugate or the pharmaceutical composition is administered in a dose and schedule effective to reduce the activity of LZTR1 protein encoded by gene LZTR1 by about 5%, 10%, 15%, 20%, 25%, 30%, 35%, 40%, 45%, 50%, 55%, 60%, 65%, 70%, 75%, 80%, 85%, 90%, or 95%.

In some instances, the drug conjugate or the pharmaceutical composition is administered in a dose and schedule effective to increase expression of a marker gene of myelination by at least 5%, 10%, 15%, 20%, 25%, 30%, 35%, 40%, 45%, 50%, 55%, 60%, 65%, 70%, 75%, 80%, 85%, 90%, or 95%. Schwann cells exist in different developmental stages or wound repair phases, which express different Schwann cell-specific markers. Accordingly, in specific instances, the drug conjugate or the pharmaceutical composition is administered in a dose and schedule effective to increase expression of a marker gene of Schwann cell precursors by at least 5%, 10%, 15%, 20%, 25%, 30%, 35%, 40%, 45%, 50%, 55%, 60%, 65%, 70%, 75%, 80%, 85%, 90%, or 95%. In specific instances, the drug conjugate or the pharmaceutical composition is administered in a dose and schedule effective to increase expression of a marker gene of immature Schwann cell by at least 5%, 10%, 15%, 20%, 25%, 30%, 35%, 40%, 45%, 50%, 55%, 60%, 65%, 70%, 75%, 80%, 85%, 90%, or 95%. In specific instances, the drug conjugate or the pharmaceutical composition is administered in a dose and schedule effective to increase expression of a marker gene of myelinating Schwann cell by at least 5%, 10%, 15%, 20%, 25%, 30%, 35%, 40%, 45%, 50%, 55%, 60%, 65%, 70%, 75%, 80%, 85%, 90%, or 95%. In specific instances, the drug conjugate or the pharmaceutical composition is administered in a dose and schedule effective to increase expression of a marker gene of non-myelinating Schwann cell by at least 5%, 10%, 15%, 20%, 25%, 30%, 35%, 40%, 45%, 50%, 55%, 60%, 65%, 70%, 75%, 80%, 85%, 90%, or 95%. In some cases, the drug conjugate or the pharmaceutical composition is administered in a dose and schedule effective to increase expression of one or more the markers, including but are not limited to, S100, p75^{NTR}, Sox10, Sox2, GAP43, NCAM, Krox20, Oct6, MBP, and MPZ, at least by 5%, 10%, 15%, 20%, 25%, 30%, 35%, 40%, 45%, 50%, 55%, 60%, 65%, 70%, 75%, 80%, 85%, 90%, or 95%.

### Certain Terminology

The term “disease-associated molecule,” as used herein, generally refers to a molecule that is directly or indirectly associated with the pathogenesis, progression, prolonging, or recurrence of a disease. In some cases, the abnormal expression level of the disease-associated molecule is associated with the pathogenesis, progression, prolonging, or recurrence of the disease. In some cases, the abnormal activity of the disease-associated molecule is associated with the pathogenesis, progression, prolonging, or recurrence of the disease.

The term “complementary,” as used herein, generally refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some instances, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

The term “conservative amino acid substitution,” as used herein, generally refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The term “antibody,” as used herein, generally refers to a polypeptide that includes at least one immunoglobulin variable domain or at least one antigenic determinant, e.g., paratope that specifically binds to an antigen. In some instances, an antibody is a full-length antibody. In some instances, an antibody is a chimeric antibody. In some instances, an antibody is a humanized antibody. However, in some instances, an antibody is a Fab fragment, a F(ab′) 2 fragment, a Fv fragment or an scFv fragment. In some instances, an antibody is a nanobody derived from a camelid antibody or a nanobody derived from shark antibody. In some instances, an antibody is a diabody. In some instances, an antibody comprises a framework having a human germline sequence. In other instances, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some instances, an antibody comprises a heavy (H) chain variable region (abbreviated herein as VH), and/or a light (L) chain variable region (abbreviated herein as VL). In some instances, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some instances, the heavy chain of an antibody described herein can be an alpha (α), delta (Δ), epsilon (ε), gamma (γ) or mu (μ) heavy chain. In some instances, the heavy chain of an antibody described herein can comprise a human alpha (α), delta (Δ), epsilon (ε), gamma (γ) or mu (μ) heavy chain. In a particular instance, an antibody described herein comprises a human gamma 1 CH1, CH2, and/or CH3 domain. In some instances, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some instances, the VH domain comprises an amino acid sequence that is at least 70%, 75%, 80%, 85%, 90%, 95%, 98%, or at least 99% identical to any of the variable chain constant regions provided herein. In some instances, an antibody is modified, e.g., modified via glycosylation, phosphorylation, sumoylation, and/or methylation. In some instances, an antibody is a glycosylated antibody, which is conjugated to one or more sugar or carbohydrate molecules. In some instances, the one or more sugar or carbohydrate molecule are conjugated to the antibody via N-glycosylation, O-glycosylation, C-glycosylation, glypiation (GPI anchor attachment), and/or phosphoglycosylation. In some instances, the one or more sugar or carbohydrate molecule are monosaccharides, disaccharides, oligosaccharides, or glycans. In some instances, the one or more sugar or carbohydrate molecule is a branched oligosaccharide or a branched glycan. In some instances, the one or more sugar or carbohydrate molecule includes a mannose unit, a glucose unit, an N-acetylglucosamine unit, an N-acetylgalactosamine unit, a galactose unit, a fucose unit, or a phospholipid unit. In some instances, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

The term “covalent,” as used herein, generally refers to a characteristic of two or more molecules being linked together via at least one covalent bond. In some instances, two molecules can be covalently linked together by a single bond, e.g., a disulfide bond or disulfide bridge, that serves as a linker between the molecules. However, in some instances, two or more molecules can be covalently linked together via a molecule that serves as a linker that joins the two or more molecules together through multiple covalent bonds. In some instances, a linker may be a cleavable linker. However, in some instances, a linker may be a non-cleavable linker.

The term “specifically binds,” as used herein, generally refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting Schwann cells, through binding to the antigen, as described herein. In some instances, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less.

The term “(poly) peptide” or “protein” as used herein, are used interchangeably, and generally refers to a series of amino acid residues joined by peptide bonds (i.e., a polymer of amino acids) and include modified amino acids (e.g., phosphorylated, glycated, glycosylated, etc.) and amino acid analogs. Illustrative polypeptides or proteins include gene products, naturally occurring proteins, homologs, paralogs, fragments and other equivalents, variants, and analogs of the above.

The terms “subject,” “individual,” “mammal”, and “patient,” as used herein, used interchangeably and generally refer to may be and refer to humans, as well as non-human mammals (e.g., non-human primates, canines, equines, felines, porcines, bovines, ungulates, lagomorphs, and the like). In various instances, the subject can be a human (e.g., adult male, adult female, adolescent male, adolescent female, male child, female child) under the care of a physician or other health worker in a hospital, as an outpatient, or other clinical context. In certain instances, the subject may not be under the care or prescription of a physician or other health worker.

The term “linker,” as used herein, generally refers to is a molecule that is used to join two or more molecules. In certain instances, the linker is capable of forming covalent bonds to both molecule(s). Suitable linkers include, but are not limited to, straight or branched-chain carbon linkers, heterocyclic carbon linkers, or peptide linkers.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

The term “a,” “an” and “the” include plural referents unless the content clearly dictates otherwise.

The term “about,” particularly in reference to a given quantity, is meant to encompass deviations of plus or minus ten percent.

While various instances of the disclosure have been shown and described herein, it will be obvious to those skilled in the art that such instances are provided by way of example only. Numerous variations, changes, and substitutions may occur to those skilled in the art without departing from the disclosure. It should be understood that various alternatives to the instances of the disclosure described herein may be employed.

### EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. Efforts have been made to ensure accuracy with respect to numbers used (e.g., amounts, percentages, etc.), but some experimental errors and deviations should be accounted for.

### Example 1: Evaluate Targeted Delivery In Vitro and In Vivo

A conjugate of ML-LBP21 and GFP is generated.

Nucleic acid sequence of ML-LBP21 is synthesized and introduced into the bacterial expression system under the control of a lac promoter having a 6×His tag at the C-terminal end, with and without Green fluorescent Protein (GFP), and later transformed into BL21 E. coli. Upon induction of IPTG (0.1 mM, at 26° C. for 16 hr), cells are centrifuged and collected. Cells are lysed using sonication and the soluble fraction collected. Cell supernatants are further purified on a HisTrap column, followed by extensive wash and elution using imidazole. The purified protein is later evaluated for purity by SDS-PAGE, analytical SEC. ML-LBP21's binding affinity to the leprosy receptor laminin 2 is also evaluated.

The delivery capability of the conjugates of ML-LBP21 and GFP to Schwann cells is first tested by incubating the conjugates with a primary Schwann cells or cell line, such as mouse Schwann cell line MSC80 which is used as a system for neuropathological studies.

In order to determine whether the ML-LBP21-GFP conjugates or another detectable Tag is targeted delivered into Schwann cells, ML-LBP21-GFP conjugates and primary cells or/and MSC80 cells are co-incubated. As a negative control, the cells are incubated with GFP alone. As a positive control, the primary or MSC80 cells are incubated with GFP with the assistance of Lipofectamine reagents (Thermo Fisher). After incubation, the number of GFP positive cells are detected by a flow cytometer.

The total fluxes of GFP positive cells are detected. The number of fluorescence detected in the group of primary cells or/and MSC80 cells incubated with ML-LBP21-GFP conjugates is higher than the ones incubated with GFP alone, and is comparable to the ones incubated with GFP with lipofectamine reagents. This proves that ML-LBP21 promotes Schwann cell delivery.

In order to verify that the uptake of ML-LBP21-GFP conjugates into primary cells or/and MSC80 cells is indeed mediated by ML-LBP21, a competitive uptake assay is carried out. After pre-incubating primary cells or/and MSC80 cells with ML-LBP21 peptides at a range of concentrations, the cells are treated with ML-LBP21-GFP conjugates, at the same time, GFP alone is used as a control. The amount of ML-LBP21-GFP conjugates detected in Schwann cells is gradually reduced with an increasing amount of ML-LBP21 peptides. However, the random low uptake of GFP into the cells is not affected by the pre-incubation of ML-LBP21 peptides. This result shows that the ML-LBP21-GFP conjugates enter Schwann cells through a ML-LBP21-mediated process.

Next, the conjugates of ML-LBP21 and GFP or another detectable Tag are injected into wild-type mice, and mice are sacrificed at different time points for the detection of GFP/Tag signals in different tissues and organs, as well as isolated sciatic nerves. GFP/Tag signals are more enriched in the isolated nerves. A further examination of the isolated nerves is carried out by a co-staining of cellular markers that distinguish neurons (axons) and Schwann cells. GFP/Tag signals are enriched in Schwann cells.

Similarly, ML-LBP21 is replaced with a targeting moiety that binds to gliomedin, and the above-described experiments are adopted to test the delivery system targeting gliomedin.

### Example 2: Evaluate Targeted Delivery of an siRNA Molecule In Vitro and In Vivo

A conjugate of an siRNA targeting a gene (e.g., a house keeping gene) and a targeting moiety (e.g., a ligand, a carbohydrate ligand, ML-LBP21, an anti-gliomedin antibody) is generated. The double stranded RNA (dsRNA) is conjugated to the targeting moiety (e.g., a ligand, a carbohydrate ligand, ML-LBP21, an anti-gliomedin antibody).

In order to determine whether the ML-LBP21-siRNA conjugates is successfully delivered into Schwann cells, ML-LBP21-siRNA conjugates are contacted to the primary Schwann cells or/and MSC80 cells. As a negative control, the primary cells or/and MSC80 cells are contacted with the naked siRNA molecules. As a positive control, the primary and/or MSC80 cells are incubated with GFP with the assistance of RNAiMax transfection reagents (Thermo Fisher). After incubation, RNAs of cells are collected and quantified by qPCR. The mRNA quantity of the target gene in the cells incubated with ML-LBP21-siRNA conjugates is lower than the ones incubated with siRNA alone, and is comparable to the one with siRNA with the RNAiMax transfection reagents.

Alternatively, the cells are genetically modified to express the target of siRNA labeled with Ruby3. After incubation, the Ruby3 signal is reduced when compared to siRNA alone, and is comparable to the one with siRNA with the RNAiMax transfection reagents.

The above experiments prove that ML-LBP21 promotes delivering the siRNA into Schwann cell.

### Example 3: Evaluate the Internalization of Targeting Moieties in Schwann Cells In Vitro

In order to identify antibodies that can bind to and get internalized specifically into Schwann cells, several antibodies that recognized extracellular epitopes of Schwann cell-expressed proteins were tested: mouse anti-myelin-associated glycoprotein (MAG) antibody mAb513 (MAB1567, Merck), rabbit anti-gliomedin antibody (raised against the olfactomedin domain of rat gliomedin), mouse anti-gliomedin antibody (mAb94, generated against residues 273-287 of rat gliomedin protein, or binding residues 273-287 of rat gliomedin protein (CVIPNDDTLVGRA)), and mouse anti-nectin-like protein 4 (Necl4, also known as Cadm4) antibody mAb244/5 (NeuroMAB). These antibodies were first pre-labeled with cy3-conjugated anti-mouse antibodies (red). For the rabbit-sourced antibodies, Cy™3 AffiniPure Donkey Anti-Rabbit IgG (H+L) antibody from Jackson ImmunoResearch Inc. (711-165-152) was used; for mouse-sourced antibodies, Cy™3 AffiniPure Donkey Anti-Mouse IgG (H+L) antibody from Jackson ImmunoResearch Inc. (715-165-151) was used. Live rat Schwann cell cultures were then incubated with these antibodies for 30 minutes, followed by washes and incubation in growth medium for additional 48 hours in incubators. Afterwards, the cell cultures were fixed and stained with an anti-mouse 488 antibody (green) without cell permeabilization. Therefore, yellow labeling showed antibody molecules that were found outside of the cells whereas red labeling shows molecules found inside of the cells.

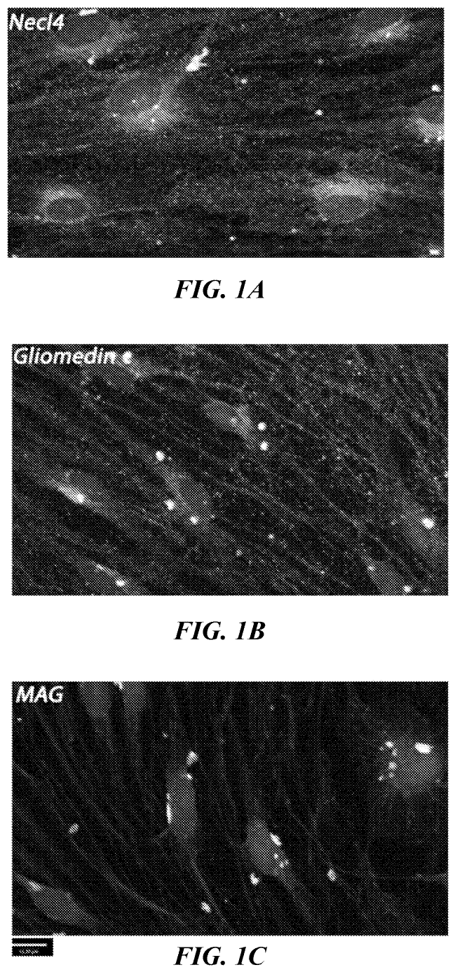

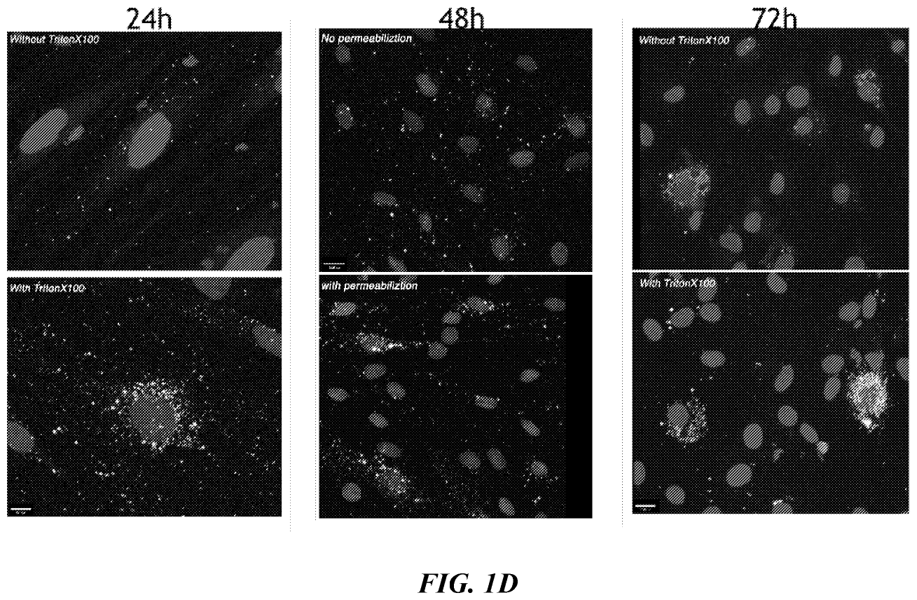

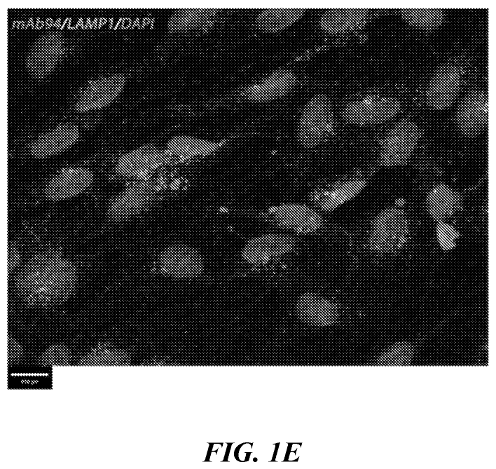

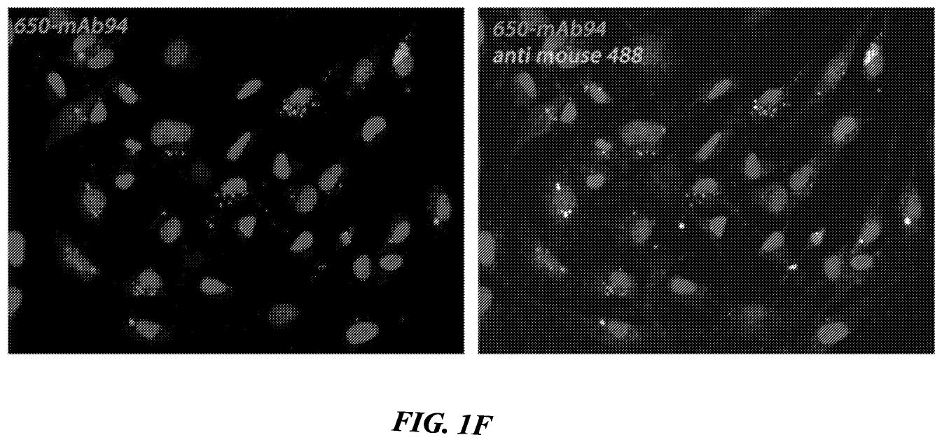

As shown in FIG. 1A and FIG. 1B, anti-Necl4 antibody (mAb244/5) and anti-gliomedin antibody (mAb94) could bind to and effectively internalize into the Schwann cells. However, FIG. 1C shows anti-MAG antibody didn't internalize into Schwann Cells.

Anti-gliomedin antibody (mAb94) was further investigated for its internalization capability. Rat Schwann cells were cultured on PLL-coated coverslips and were incubated with anti-gliomedin antibody (mAb94) that was pre-incubated with cy3-conjugated anti mouse antibody (red) for 30 minutes at 37° C. The cells were then washed and incubated in growth medium for the indicated amount of time in FIG. 1D (i.e. 24 h, 48 h, or 72 h). Afterwards, the cells were fixed. Fixed cells were labelled with anti-mouse 488 antibodies in the presence or absence of 0.1% Triton X100 in the antibody solution. Triton is a detergent that permeabilizes cell membranes, so cell membrane remained intact without Triton, and only anti-gliomedin antibody (mAb94) molecules found outside of the cells would be co-labeled with 488 antibody (green) along with the red labeling, while those Ab molecules that had been internalized would be labeled only in red. In other words, the experiments without cell permeabilization differentiated between extra- and intra-cellular molecules, while the ones with cell permeabilization labeled the entire antibody population. As shown in FIG. 1D, while at 24 hours about half of the molecules were still co-labeled with 488 without permeabilization, half of the molecules were found outside of the cells. At 72 hours, a significant portion of the antibody population was internalized and hence was not co-labeled with 488 without cell permeabilization. In other words, the co-localization of the green and red signals only in the presence of TritonX but not in the absence of Triton X indicating that the antibodies were internalized into the cells (e.g., 72 hr).

Next, the sub-cellular localization of the internalized anti-gliomedin antibody (mAb94) at 72 hours was investigated. As shown in FIG. 1E, a partial co-localization of anti-gliomedin antibody (mAb94) with a lysosomal marker Lamp1 indicated that anti-gliomedin antibody (mAb94) entered lysosomes.

Furthermore, in order to test the internalization of targeting moieties without the need of preincubating with labeling secondary antibodies, Cyanine5 NHS was covalently conjugated to and label anti-gliomedin antibody (mAb94) and a control mouse IgG antibody. Antibody samples in PBS were mixed with 10× solution of NaHCO₃ (1M pH; 8.3) and Cyanine5 NHS (Lumiprobe cat #43020), which was prepared as stock solution (2.66 mg/ml DMSO) at molar ratio for the fluorescence dye and the antibodies of 7:1. The tubes with the reaction mixture were kept in darkness and allowed to incubate for 1 hr at RT under continuous mixing. The labeling reaction was stopped after 1 hr by addition of 5 ml of 1M Tris-HCl pH 7.1 and dialyzed against PBS buffer.

Rat Schwann cells were incubated for 30 minutes at 37° C. with the covalently labeled anti-gliomedin antibody (mAb94) and with control IgG antibody (red), followed by 24 hour incubation in growth medium. Cells were fixed and stained with anti-mouse 488 without cell permeabilization to label extracellular antibody molecules. As shown in FIG. 1F, the Cy3 labeled mAb94 antibody did not co-localize with 488 signals, suggesting that it was internalized into the Schwann cells.

### Example 4: Evaluate the Specificity of Anti-Gliomedin Antibody (mAb94)

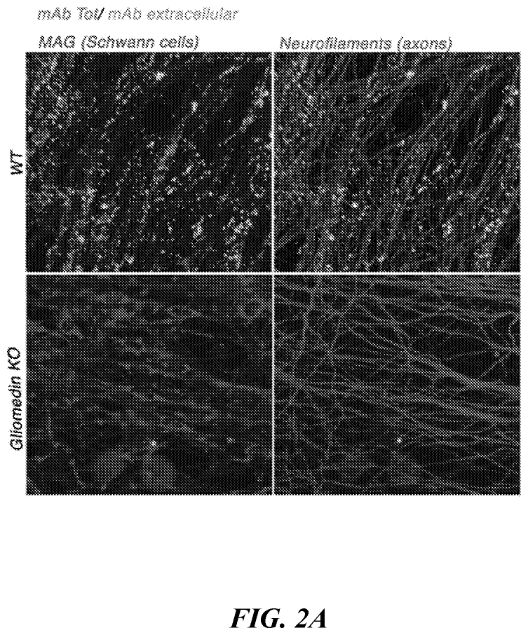

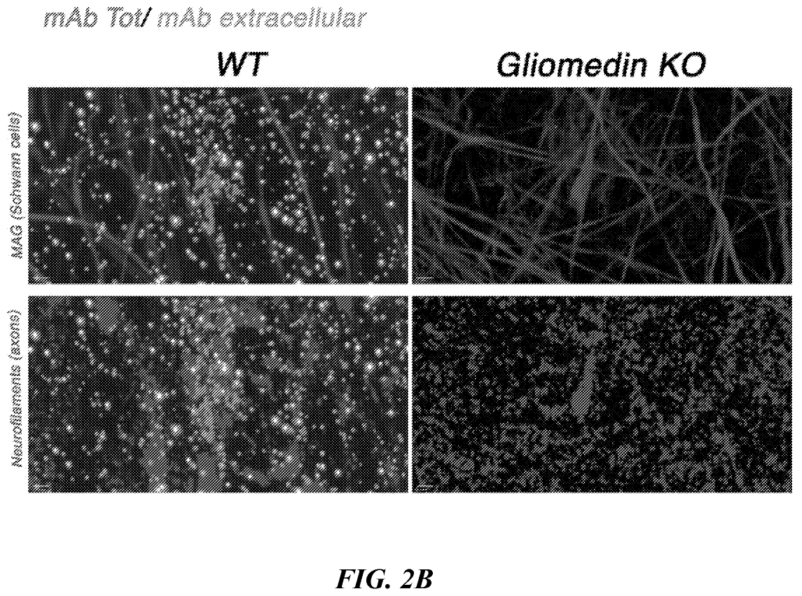

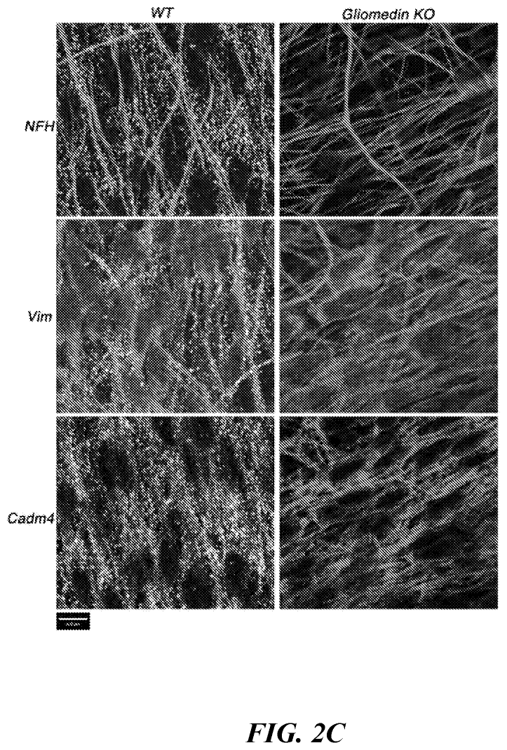

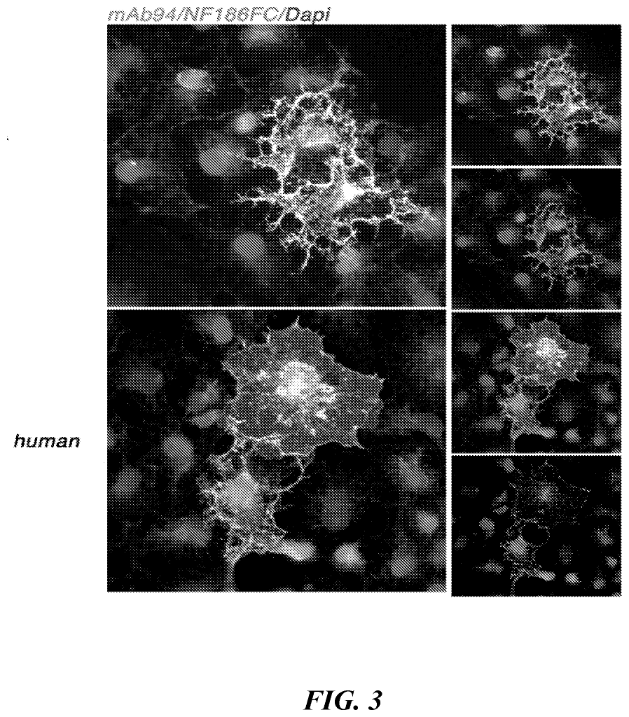

Similar incubation settings as disclosed in Example 3 were applied to primary cultures containing sensory neurons and Schwann cells, generated from dorsal root ganglia (DRG) that was isolated from either wildtype (“WT”) or gliomedin null mouse embryos (“KO”). Anti-gliomedin antibody (mAb94) was added to the cell cultures and cultures were further incubated. Anti-mouse 488 antibodies (green) was applied without membrane permeabilization followed by a second fixation and labeling with either a Schwann cells marker antibody (“MAG”) or a neuronal marker Ab (“neurofilaments”). As shown in FIG. 2A and FIG. 2B, anti-gliomedin antibody (mAb94) internalized specifically to Schwann cells in the mixed DRG neuron preparation. No signals from anti-gliomedin antibody (mAb94) were detected in the receptor-null DRG cultures.

The cell-type specificity was further confirmed. DRG myelinating cultures isolated from E13 mouse embryos contained 3 cell types: Schwann cells, sensory DRG neurons, and fibroblasts. Anti-gliomedin antibody (mAb94) antibodies (green) were applied to live DRG cultures for 30 minutes at 37° C. Afterwards, the cultures were fixed and stained with marker antibodies (red) to differentiate neurons (neurofilaments “NFH”), fibroblasts (Vimentin “Vim”), and Schwann cells (“Cadm4”). The signals from mAb94 antibody only co-localized with the Schwann cell marker antibody, and was not detected at all in gliomedin null cultures indicating that the antibody recognized only gliomedin and did not cross react with other proteins (see FIG. 2C).

Furthermore, anti-gliomedin antibody (mAb94) recognized gliomedin from different species. COS7 cells were transfected with rat or human gliomedin. Cells were allowed to bind the extracellular domain of NF186 (NF186FC, red). Cells were later washed, fixed, and labeled with anti-gliomedin antibody (mAb94). mAb94 antibody bound both rat and human gliomedin.

### Example 5: Evaluate the Internalization of Anti-Gliomedin Antibody (mAb94) into Schwann Cells In Vivo

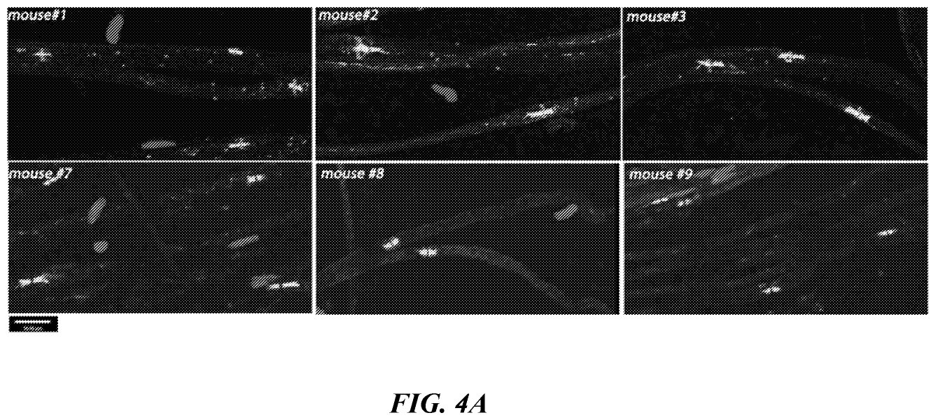

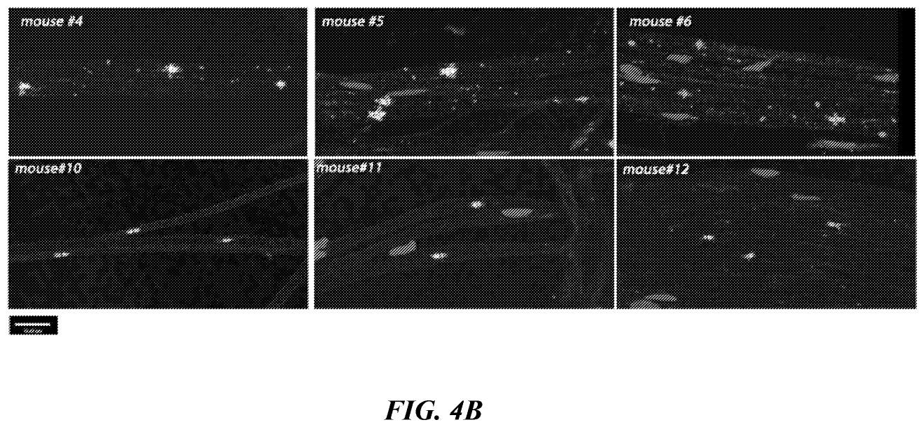

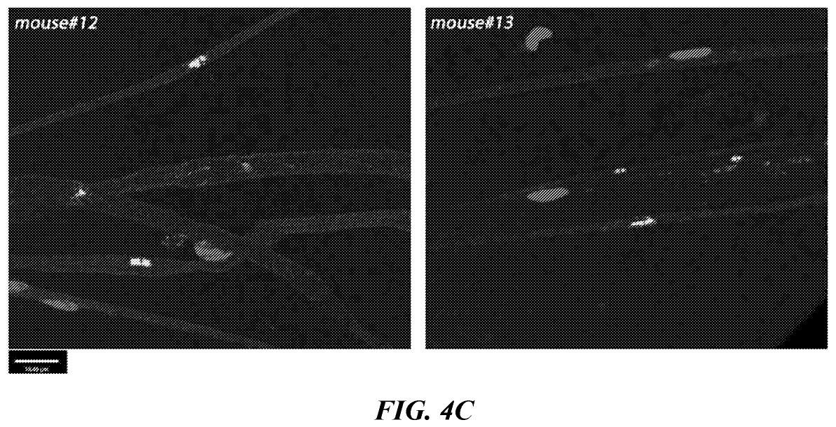

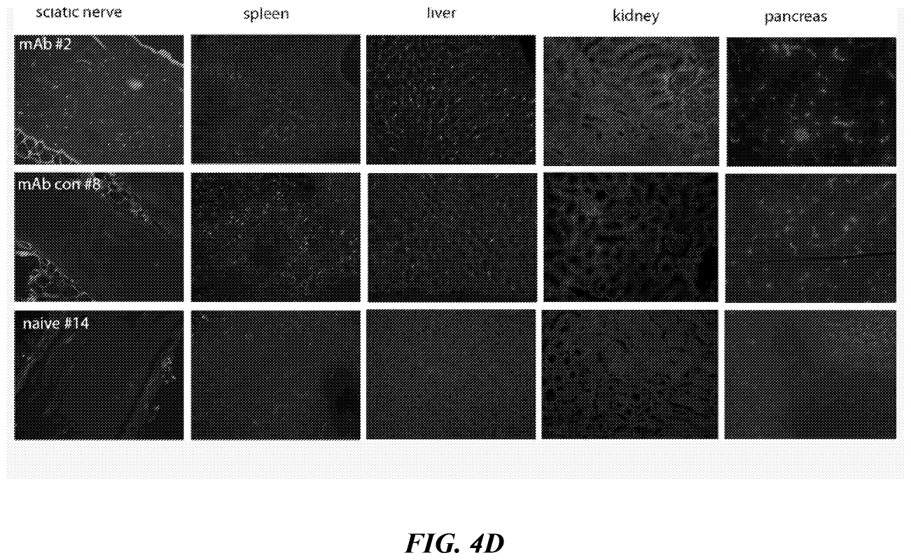

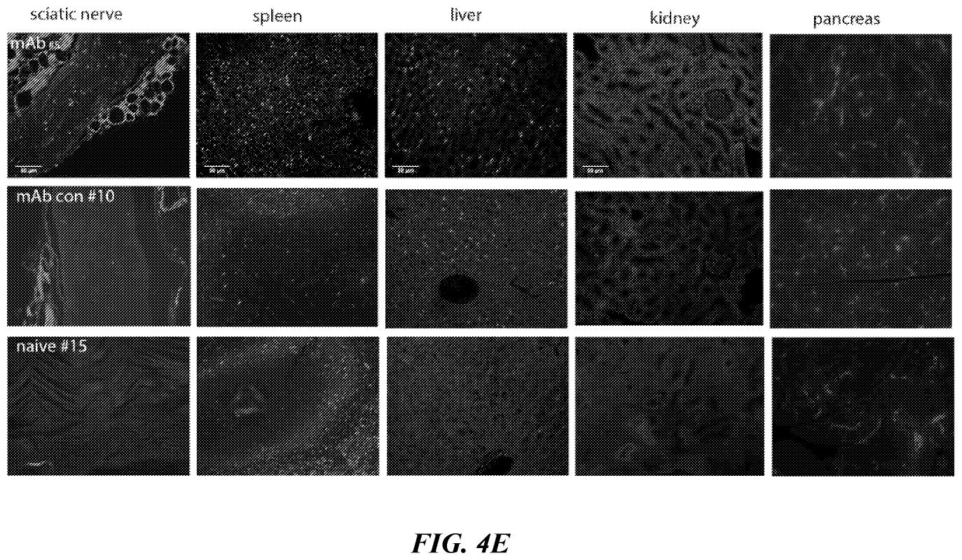

To test the targeting of anti-gliomedin antibody (mAb94) to Schwann cells in vivo, mice were injected via tail veins with 150 μg of 650-mAb94 antibody (mice #1-3 and #4-6), control 650-IgG (#7-9 and #10-12), or nothing (mice #13-15). Mice were sacrificed 24 or 48 hours post injection. Sciatic nerves were collected, fixed, and spread (teased) at a single fiber imaging resolution. Teased samples were labeled with an anti-caspr antibody (green) which labeled the node of Ranvier of myelinated nerve fibers. In the 650mAb94-injected mice nerves, anti-gliomedin antibody (mAb94) (red) was strongly detected at nodes of Ranvier and at clusters along the entire myelin units (see mice #1-3 for 24 hr in FIG. 4A and mice #4-6 for 48 hr in FIG. 4B). The data indicated that mAb94 antibody penetrated the nerve and bound gliomedin on Schwann cells. In contrast, no significant signal was detected in the nerves of the control-injected mice (see mice #7-9 for 24 hr in FIG. 4A and mice #10-12 for 48 hr in FIG. 4B) or mice with no injection (see e.g., mouse #13 in FIG. 4C). It was clear that only anti-gliomedin antibody (mAb94) showed binding to myelinated nerves at the node of Ranvier and along the Schwann cell internode. However, the control IgG antibody did not show any specific signals and had similarly minimum staining as the un-injected mouse.

Therefore, these data suggested that anti-gliomedin antibody (mAb94) was able to cross blood nerve barrier, to reach the nerves, and to internalize into Schwann cells.

Furthermore, the distribution of anti-gliomedin antibody (mAb94) in different organs was evaluated using fluorescent microscopy.

Samples (n=75) of different organs from the 15 mice were harvested following fluorescent labeling, fixed in 4% paraformaldehyde (PFA) and kept in the fixative for 48 hours for further fixation. The tissues were trimmed, put in embedding cassettes, and processed for paraffin embedding with a protocol shown below. Four cassettes were prepared per animal.

Tissue processor was modified to a very short protocol in order to preserve the fluorescent signal. 1. Formalin for 10 min at 37° C.; 2. Processing water for 2 min; 3. Ethanol 70% for 5 min at 45° C.; 4. Ethanol 80% for 5 min at 45° C.; 5. Ethanol 95% for 5 min at 45° C.; 6. Ethanol 100% for 5 min at 45° C.; 7. Ethanol 100% for 5 min at 45° C.; 8. Ethanol 100% for 5 min at 45° C.; 9. Xylene for 3 min at 45° C.; 10. Xylene for 3 min at 45° C.; 11. Xylene for 5 min at 45° C.; 12. Paraffin for 5 min at 65° C.; 13. Paraffin for 5 min at 65° C.; and 14. Paraffin for 10 min at 65° C.

Paraffin sections (4 microns thick) were cut and put on glass slides. The sections were evaluated for the fluorescent marker anti-gliomedin antibody (mAb94) carried using a fluorescent microscope. Pictures shown in FIG. 4D and FIG. 4F were taken using Olympus microscope (BX60, serial No. 7D04032) equipped with microscope's Camera (Olympus DP73, serial No. OH05504) at objective magnification of X10 and X20. The number of fluorescent labelled cells at objective magnification of X40 was scored with a semi-quantitative system: Grade 0: No positive labelled cells detected; Grade 1:1-30 labelled cells detected; Grade 2:31-100 labelled cells detected; Grade 3:101-150 labelled cells detected; Grade 4: >150 labelled cells detected. Table 1 summarizes the specific localization and distribution of detected antibodies.

TABLE 1
Evaluation of anti-gliomedin antibody
(mAb94) labeled cells for each mouse

| | | | Fluorescence | Local- | |
|---|---|---|---|---|---|
| Group | sample | | score | ization of | |
| No. | No. | Organ | (paraffin) | the signal | Comments |
| 1 | 1 | Liver | 4 | In sinusoid | Artifact |
| | 24 hr | | | only | |
| | | Kidneys | 0 | | |
| | | pancreas | 0 | | |
| | | spleen | 0 | | |
| | | Peripheral | 2 | In nerve | |
| | | nerves (PNS) | | fibers | |
| | 2 | Liver | 4 | In sinusoid | Artifact |
| | 24 hr | | | only | |
| | | Kidneys | 0 | | |
| | | pancreas | 0 | | |
| | | spleen | 0 | | |
| | | Peripheral | 2 | In nerve | |
| | | nerves (PNS) | | fibers | |
| | 3 | Liver | 4 | In sinusoid | Artifact |
| | 24 hr | | | only | |
| | | Kidneys | 0 | | |
| | | pancreas | 0 | | |
| | | spleen | 0 | | |
| | | Peripheral | 0 | | |
| | | nerves (PNS) | | | |
| | 4 | Liver | 4 | In sinusoid | Artifact |
| | 48 hr | | | only | |
| | | Kidneys | 0 | | |
| | | pancreas | 0 | | |
| | | spleen | 0 | | |
| | | Peripheral | 4 | In nerve | |
| | | nerves (PNS) | | fibers | |
| | 5 | Liver | 4 | In sinusoid | Artifact |
| | 48 hr | | | only | |
| | | Kidneys | 0 | | |
| | | pancreas | 0 | | |
| | | spleen | 0 | | |
| | | Peripheral | 4 | In nerve | |
| | | nerves (PNS) | | fibers | |
| | 6 | Liver | 2 | In sinusoid | Artifact |
| | 48 hr | | | only | |
| | | Kidneys | 0 | | |
| | | pancreas | 0 | | |
| | | spleen | 0 | | |
| | | Peripheral | 1 | In nerve | |
| | | nerves (PNS) | | fibers | |
| 2 | 7 | Liver | 0 | | |
| | 24 hr | Kidneys | 0 | | |
| | | pancreas | 0 | | |
| | | spleen | 0 | | |
| | | Peripheral | 0.5 | | ? |
| | | nerves (PNS) | | | |
| | 8 | Liver | 2 | In sinusoid | Artifact |
| | 24 hr | | | only | |
| | | Kidneys | 0 | | |
| | | pancreas | 0 | | |
| | | spleen | 0 | | |
| | | Peripheral | 0 | | |
| | | nerves (PNS) | | | |
| | 9 | Liver | 2 | In sinusoid | Artifact |
| | 24 hr | | | only | |
| | | Kidneys | 0 | | |
| | | pancreas | 0 | | |
| | | spleen | 0 | | |
| | | Peripheral | 0 | | |
| | | nerves (PNS) | | | |
| | 10 | Liver | 2 | In sinusoid | Artifact |
| | 48 hr | | | only | |
| | | Kidneys | 0 | | |
| | | pancreas | 0 | | |
| | | spleen | 0 | | |
| | | Peripheral | 0 | | |
| | | nerves (PNS) | | | |
| | 11 | Liver | 4 | In sinusoid | Artifact |
| | 48 hr | | | only | |
| | | Kidneys | 0 | | |
| | | pancreas | 0 | | |
| | | spleen | 0 | | |
| | | Peripheral | 0 | | |
| | | nerves (PNS) | | | |
| | 12 | Liver | 2 | In sinusoid | Artifact |
| | 48 hr | | | only | |
| | | Kidneys | 0 | | |
| | | pancreas | 0 | | |
| | | spleen | 0 | | |
| | | Peripheral | 0 | | |
| | | nerves (PNS) | | | |
| 3 | 13 | Liver | 1 | In sinusoid | Artifact |
| | | | | only | |
| | | Kidneys | 0 | | |
| | | pancreas | 0 | | |
| | | spleen | 0 | | |
| | | Peripheral | 0 | | |
| | | nerves (PNS) | | | |
| | 14 | Liver | 0 | | |
| | | Kidneys | 0 | | |
| | | pancreas | 0 | | |
| | | spleen | 0 | | |
| | | Peripheral | 0 | | |
| | | nerves (PNS) | | | |
| | 15 | Liver | 0 | | |
| | | Kidneys | 0 | | |
| | | pancreas | 0 | | |
| | | spleen | 0 | | |
| | | Peripheral | 0 | | |
| | | nerves (PNS) | | | |

In general, a clear Cy5 signal was found in some of the organs of groups 1 and 2 mainly. In the peripheral nerves a clear signal was found in the nerves fibers that was spread diffusely along the fiber. In the livers, a strong signal was found in the sinusoids only, however this reaction was found in the negative control and naïve animals as well. In the spleens, a signal was also found in the red pulp, but a more detailed examination revealed autofluorescence due to erythrocytes. In the kidney and pancreas, no signal was found at all.

The nerves from group 1 (anti-gliomedin antibody (mAb94)) were mostly positive for the signal (5/6) and a stronger signal was recorded in 48 hours after injection compared to 24 hours. In group 2 (Isotype control), one animal showed a very weak signal, possibly because the blocking process of the Isotype Ab was not complete in the animal. In group 3 (naïve), animals were found negative.

To summarize, fluorescent labeled anti-gliomedin antibody (mAb94) was intravenously injected in 12 animals. A marked and reliable signal was only observed in the peripheral nerves. The signal from 48 hours after the injection was stronger compared to the signal from 24 hours.

### Example 6: Evaluate the Internalization of Anti-Laminin Alpha-2 Antibody into Schwann Cells

An antibody that binds to laminin alpha-2 is further investigated for its binding and internalization capability to Schwann cells.

Antibody candidates, without excluding other possible ones, may be antibody ABIN7439129 which targets amino acids 2901-3106 of laminin alpha-2, an antibody against laminin-α2 G5 (2D4) from Abnova (Taipei, Taiwan), or laminin alpha-2 monoclonal antibody (Thermo Fisher, #CL3450).

In order to evaluate the potential of the tested anti-laminin alpha-2 antibody (e.g., Invitrogen CL3450) to bind to Schwann cells, rat sciatic nerve sections are used. Sciatic nerves are excised and fixed in 4% PFA for 20 minutes at room temperature (RT). Nerves are washed with PBS and spread (teased) on glass slides to achieve a single fiber separation. The teased preparations are allowed to dry for two hours and stored at −20° C. until use. For immunofluorescent labeling, slides are incubated in blocking solution (PBS, 5% normal goat serum, 0.5% TritonX100) for 1 hour at RT. Slides are then washed with PBS and incubated with primary antibodies: the tested anti-laminin alpha-2 antibody along with an anti-caspr antibody to label myelinated axons at the node of Ranvier overnight at 4° C. The primary antibodies are diluted in antibody solution (PBS, 5% normal goat serum, 0.1% Tritonx100). Slides are subsequently washed with PBS and incubated with secondary antibodies diluted in antibody solution for 45 minutes at RT. Slides are then washed with PBS and mounted with e.g., fluoromount-G (Invitrogen). Signals from the tested anti-laminin alpha-2 antibody is analyzed in relation to structures such as the nodes of Ranvier and the basal lamina covering the myelinating Schwann cell.

The in vitro internalization capacity of the tested anti-laminin alpha-2 antibody is also investigated. Neuronal cells (including Schwann cells) are freshly dissected from sciatic nerve of mice, cultured on PLL-coated coverslips, and incubated with the pre-labeled anti-laminin alpha-2 antibody. The neuronal cells are then washed and incubated in growth medium for the different amounts of time (e.g. 24 h, 48 h, or 72 h). Afterwards, the neuronal cells are fixed, and labelled with secondary antibodies that recognize the tested anti-laminin-2 antibody in the presence or absence of 0.1% Triton X-100 for different permeabilization conditions. Under different incubation durations without permeabilization, the percentages of the tested anti-laminin alpha-2 antibody that is not co-labeled with the secondary antibody are compared.

Furthermore, the cell-type specificity of the tested anti-laminin-2 antibody is tested. Similar incubation settings are applied to primary cultures containing sensory neurons, fibroblasts, and Schwann cells, possibly generated from DRG that is isolated from either wildtype or laminin-2 null mouse embryos. Cell type markers, such as neurons (neurofilaments), fibroblasts (Vimentin), and Schwann cells (MAG), are used to differentiate different cell types.

In addition, the in vivo internalization capability of the tested anti-laminin alpha-2 antibody is further investigated. Mice are grouped and injected with labeled anti-laminin alpha-2 antibody, control labeled IgG, or nothing. Mice are sacrificed 24 or 48 hours post injection. Sciatic nerves are collected, fixed, and spread (teased) at a single fiber imaging resolution. Teased samples are labeled with an anti-caspr antibody which labels the node of Ranvier of myelinated nerve fibers. The signals from the labeled anti-laminin-2 antibody are detected.

### Example 7: Evaluate the Internalization of Anti-Cadm4/Necl4 Antibody into Schwann Cells

An antibody that binds to Cadm4/Necl4 is further investigated for its internalization capability to Schwann cells. The antibody mAb244/5 (NeuroMAB) that has shown promising internalization results in FIG. 1A is further used. Other antibody candidates, without excluding other possible ones, may be Necl4-Fc antibody from Eshed et al., Neuron, volume 47, issue 2, p215-229, anti-SynCAM4 Antibody (Biolegand, #833302), or IGSF4C/SynCAM4 Antibody (rndsystems, #MAB41642). Schwann cells are cultured on PLL-coated coverslips and are incubated with the pre-labeled anti-Cadm4/Necl4 antibody. The cells are then washed and incubated in growth medium for the different amounts of time (e.g. 24 h, 48 h, or 72 h). Afterwards, the cells are fixed and are labelled with secondary antibodies that recognize the tested anti-Cadm4/Necl4 antibody in the presence or absence of 0.1% Triton X-100 for different permeabilization conditions. Under different incubation durations without permeabilization, the percentages of the tested anti-Cadm4/Necl4 antibody that is not co-labeled with the secondary antibody are compared.

Furthermore, the cell-type specificity of the tested anti-Cadm4/Necl4 antibody is tested. Similar incubation settings are applied to primary cultures containing sensory neurons, fibroblasts, and Schwann cells, possibly generated from DRG that is isolated from either wildtype or Cadm4/Necl4 null mouse embryos. Cell type markers, such as neurons (neurofilaments), fibroblasts (Vimentin), and Schwann cells (MAG), are used to differentiate different cell types.

In addition, the internalization capability in vivo of the tested anti-Cadm4/Necl4 antibody is further investigated. Mice are grouped and injected with labeled anti-Cadm4/Nec14 antibody, control labeled IgG, or nothing. Mice are sacrificed 24 or 48 hours post injection. Sciatic nerves are collected, fixed, and spread (teased) at a single fiber imaging resolution. Teased samples are labeled with an anti-caspr antibody which labels the node of Ranvier of myelinated nerve fibers. The signals from the labeled anti-Cadm4/Necl4 antibody are detected.

It shall be understood that different aspects of the disclosure can be appreciated individually, collectively, or in combination with each other. Various aspects of the disclosure described herein may be applied to any of the particular applications disclosed herein. The compositions of matter disclosed herein in the composition section of the present disclosure may be utilized in the method section including methods of use and production disclosed herein, or vice versa.

While preferred instances of the present disclosure have been shown and described herein, it will be obvious to those skilled in the art that such instances are provided by way of example only. It is not intended that the disclosure be limited by the specific examples provided within the specification. While the disclosure has been described with reference to the aforementioned specification, the descriptions and illustrations of the instances herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the instances of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1. A drug conjugate comprising a targeting moiety conjugated to a drug molecule, wherein the targeting moiety binds to a receptor expressed on a Schwann cell to mediate targeted delivery of the drug conjugate to the Schwann cell, and wherein the drug molecule modifies expression or activity of a disease-associated molecule in the Schwann cell.

2. The drug conjugate of claim 1, wherein the receptor is selected from a group consisting of a leprosy receptor (e.g., laminin 2, P0 protein), gliomedin, or cell adhesion molecule (Cadm).

3. The drug conjugate of claim 2, wherein the receptor is gliomedin.

4. The drug conjugate of claim 2, wherein the receptor is Cadm.

5. The drug conjugate of claim 2, wherein the receptor is a leprosy receptor.

6. The drug conjugate of claim 1, wherein the targeting moiety is an antibody or antigen binding fragment thereof or a ligand molecule.

7. The drug conjugate of claim 6, wherein the antibody or antigen binding fragment thereof comprises monovalent Fab′, divalent Fab2, single-chain variable fragment (scFv), diabody, minibody, nanobody, single-domain antibody (sdAb), or camelid antibody or binding fragment or antibody-mimetic thereof.

8. The drug conjugate of claim 1, wherein the targeting moiety is a ligand molecule, and wherein the ligand molecule comprises a peptide, a glycoprotein, a carbohydrate moiety, or a synthetic small molecule.

9. The drug conjugate of claim 1, wherein the drug molecule comprises a peptide or a small molecule.

10. The drug conjugate of claim 1, wherein the drug molecule comprises a double-stranded RNAi molecule or a single-stranded antisense oligonucleotide.

11. The drug conjugate of claim 1, wherein the drug molecule is conjugated with the targeting moiety via a linker.

12. The drug conjugate of claim 11, wherein the linker is a cleavable linker or a non-cleavable linker.

13. The drug conjugate of claim 1, wherein the drug molecule to the targeting moiety ratio is 1:1, 2:1, or 1:2.

14. A pharmaceutical composition comprising: a drug conjugate of claim 1 and a pharmaceutically acceptable excipient.

15. The pharmaceutical composition of claim 14, wherein the pharmaceutical composition is formulated for parenteral, intravenous, subcutaneous, or intrasciatic injection.

16. A method of targeted delivery of a drug to a Schwann cell, comprising contacting the drug conjugate of claim 1 to the Schwann cell, wherein the drug molecule comprises the drug.

17. The method of claim 16, wherein the drug is internalized into the Schwann cell upon binding of the targeting moiety to the receptor on the Schwann cell.

18. A method of targeted delivery of a drug to a Schwann cell, comprising contacting the pharmaceutical composition of claim 14 to the Schwann cell, wherein the drug molecule comprises the drug.

19. The method of claim 18, wherein the drug is internalized into the Schwann cell upon binding of the targeting moiety to the receptor on the Schwann cell.
